(19) United States
(12) Patent Application Publication
(10) Pub. No.: US 2018/0319657 A1
(43) Pub. Date: Nov. 8, 2018

(54) Multiplex Lateral Flow Devices and Assays

(71) Applicant: BIOCIFER PTY LTD, Maroochydore, QLD (AU)

(72) Inventors: JOANNE MACDONALD, SIPPY DOWNS (AU); JIA LI, SIPPY DOWNS (AU)

(21) Appl. No.: 15/773,581

(22) PCT Filed: Nov. 4, 2016
(86) PCT No.: PCT/AU2016/000371

§ 371 Date: May 4, 2018

(30) Foreign Application Priority Data
Nov. 4, 2015 (AU) 2015904524

(51) Int. Cl. B82Y 5/00; B82Y 10/00; B82Y 15/00; G01N 33/53

(52) U.S. Cl. CPC B82Y 5/00; B82Y 10/00; G01N 33/582; G01N 33/5302; C12Q 2537/143; B82Y 15/00

(57) ABSTRACT

The present invention relates generally to the field of immunoassays. More specifically, the present invention relates to multiplex lateral flow devices (LFDs) and methods for detecting analytes using multiplex LFDs.

### INCORPORATION BY CROSS-REFERENCE

The present application claims priority from Australian provisional patent application number 2015904524 filed on 4 Nov. 2015, the entire content of which is incorporated herein by cross-reference.

### TECHNICAL FIELD

The present invention relates generally to the field of immunoassays. More specifically, the present invention relates to multiplex lateral flow devices (LFDs) and methods for detecting analytes using multiplex LFDs.

### BACKGROUND

Over the last three decades, lateral flow biosensors have emerged as popular and practical tools towards point-of-care (POC) diagnostics, and have gained a market share of more than a third of all POC tests¹. The most famous example is the home pregnancy test that was commercialised in 1988² into one of the most fundamentally successful products ever developed for onsite biomedical diagnosis. The US market alone is estimated at 20 million pregnancy tests sold per year³, and the global market estimate is approximately 1.68 billion pregnancy tests sold each year. Lateral flow devices (LFDs) are ideal candidates for low resource and point-of-care implementation due to their rapidness, simplicity and low cost, whilst retaining high diagnostic sensitivity and specificity.

Multiplexing is a critical parameter for increasing the diagnostic efficiency of LFDs and other immunoassays. The strategies that enable simultaneous analysis of multiple samples are largely dependent on the underlying diagnostic technology. Mature technologies such as enzyme-linked immunosorbent assay (ELISA) and real-time polymerase chain reaction (PCR) enable limited multiplexing in laboratory and clinical settings, but are time-consuming to perform. Contemporary methods focus on microarray-based technologies coupled with nanomaterials (e.g. magnetic nanoparticles) for detection, such as the Bio-Rad Bio-Plex® Systems⁴ and Luminex MagPix®⁵. These enable reduced sample volume and shorter detection times using high-throughput and even automated processing methods. However, all of these multiplexing technologies require highly equipped institutions with skilled technicians for operation and, therefore, preclude deployment to low resource or POC settings.

Very few commercially available LFDs employ multiplexing. The most direct LFD multiplexing strategy is to simply increase the number of test lines along the length on a single device⁶. However, such linear expansion is limited, as described by Washburn's theory which purports that the flow rate in a porous matrix is inversely proportional to the wicking distance⁷. Thus, the flow rate decreases with distance from the conjugate pad and consequently the assay time increases. In addition, the number of test lines that can be added is limited by the size of the device. Alternatively, some commercial LFDs accommodate several parallel dipsticks in a single cassette, such as the BDTM Directigen™ EZ Flu A+B kit (Franklin Lakes, N.J.)⁸, the Alere BinaxNOW® Influenza A&B Card (Orlando, Fla.)⁹, and the RAID 8 and RAID TOX (Wheeling, Ill.)¹⁰. This does not directly address multiplex expansion, as it increases reagent consumption in multiples of the single dipstick. Apart from parallelism, others have increased multiplexing in a multi-directional manner, providing more arms or zones to accommodate more test lines¹¹. However, this strategy consumes as much reagent as the parallelism strategy and increases the dimension size of the devices. In view of these and other problems faced by existing strategies, the number of entities detectable by current multiplex LFDs is typically restricted which in turn reduces diagnostic capacity.

In addition, when applied for the detection of nucleic acids only a limited number of detection molecules have been demonstrated to be effectively incorporated into nucleic acids during amplification protocols and subsequently detected via LFDs, which limits multiplexing capacity.

A need exists for improved multiplex LFDs that reduce or remove at least one of the problems noted above.

### SUMMARY OF THE INVENTION

The present invention addresses various problem/s associated with existing multiplex immunoassays and/or LFDs by expanding multiplex detection in a lateral flow system that increases the scope and efficiency of analyte detection, without consuming excess reagents. The LFDs described herein may provide at least one of the following improvements over some or all existing LFDs:
capacity to detect larger numbers of analytes via a small set of detection molecules;
discrete sets of antibody-ligand pairs for general application in LFDs that minimise cross-reactivity (false positives) and maximise sensitivity during analyte detection;
reduced dimension size/increased compactness;
reduced reagent consumption;
improved efficiency of analyte detection;
a digital-like result display powered by molecular interactions.

Without any placing any particular limitation on its scope, the present invention relates at least to the following embodiments:

Embodiment 1: A lateral flow device comprising three binding molecule populations for detection of multiple analytes, wherein each said binding molecule population:

has binding specificity for a different type of target ligand,

has less than: 10%, 5%, 4%, 3%, 2%, or 1%, cross-reactivity with a target ligand for which any other said binding molecule population has binding specificity; and

is capable of contributing to the formation of a signalling complex capable of providing a detectable signal only in the presence of a target analyte, wherein the signalling complex comprises a signalling molecule, and a member of any one of said binding molecule populations bound directly to said target ligand for which the member has binding specificity, which is in turn bound either directly or indirectly to said target analyte.

Embodiment 2: The lateral flow device according to embodiment 1, wherein each said binding molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin and biotin.

Embodiment 3: The lateral flow device according to embodiment 1 or embodiment 2, wherein each of said multiple analytes, or each said binding molecule population is immobilised within a detection zone of the device in a spatially separated arrangement.

Embodiment 4: The lateral flow device according to embodiment 3, wherein

the binding molecule that exhibits the lowest level of sensitivity in the presence of its target analyte is positioned closer to a sample application zone of the device compared to any other binding molecule population immobilised in the detection zone.

Embodiment 5: The lateral flow device according to embodiment 3, wherein

the binding molecule that exhibits the highest level of sensitivity in the presence of its target analyte is positioned furthest from a sample application zone of the device compared to any other analyte immobilised in the detection zone.

Embodiment 6: The lateral flow device according to any one of embodiments 3 to 5, wherein the spatially separated arrangement is non-linear.

Embodiment 7: The lateral flow device according to any one of embodiments 3 to 6, wherein the spatially separated arrangement is a non-linear dot or line format.

Embodiment 8: The lateral flow device according to any one of embodiments 3 to 7, wherein the spatially separated arrangement is a dot matrix format.

Embodiment 9: The lateral flow device according to any one of embodiments 3 to 8, comprising seven different detection molecule populations each immobilised within a detection zone of the device in a dot matrix format, wherein

each of three of the binding molecule populations are represented in the dot matrix by two dots per population which are equidistant or substantially equidistant from a sample application zone of the device, and

each of four of the binding molecule populations are represented in the dot matrix by one dot per population which are at different distances from a sample application zone of the device, and

all said dots are collectively arranged in a pattern forming the digit eight.

Embodiment 10: The lateral flow device according to any one of embodiments 1 to 9, wherein the three binding molecule populations are a combination shown in Table 3.

Embodiment 11: The lateral flow device according to any one of embodiments 1 to 10 comprising four binding molecule populations, wherein the four binding molecule populations are a combination shown in Table 4.

Embodiment 12: The lateral flow device according to any one of embodiments 1 to 11 comprising five binding molecule populations, wherein the five binding molecule populations are a combination shown in Table 5.

Embodiment 13: The lateral flow device according to any one of embodiments 1 to 12 comprising six binding molecule populations, wherein the six binding molecule populations are a combination shown in Table 6.

Embodiment 14: The lateral flow device according to any one of embodiments 1 to 13 comprising seven binding molecule populations, wherein the seven binding molecule populations are a combination shown in Table 7.

Embodiment 15: The lateral flow device according to embodiment 14, wherein the seven detection molecule populations are anti-digoxigenin antibodies, anti-TAMRA antibodies; anti-Texas Red antibodies, anti-dinitrophenyl antibodies, anti-Cascade Blue antibodies, either one of streptavidin or anti-biotin antibodies, and either one of anti-Dansyl antibodies or anti-Cy5 antibodies.

Embodiment 16: The lateral flow device according to any one of embodiments 1 to 15, further comprising a positive control molecule population.

Embodiment 17: The lateral flow device according to any one of embodiments 1 to 16, further comprising a capture molecule population having binding specificity for a capture ligand, wherein,

the capture molecule population and each said binding molecule population have binding specificity for different target ligands;

the capture molecule population has less than: 10%, 5%, 4%, 3%, 2%, or 1%, cross-reactivity with a target ligand for which any other said binding molecule population has binding specificity;

individual members of the capture molecule population are each bound to a signal generating molecule capable of providing said detectable signal; and

said signal complex comprises a member of the capture molecule population bound to said capture ligand which is in turn bound to said target analyte.

Embodiment 18: The lateral flow device according to embodiment 16, wherein the capture molecule population is selected from the group consisting of: anti-digoxigenin antibodies, anti-TAMRA antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, biotin, and streptavidin.

Embodiment 19: The lateral flow device according to embodiment 17 or embodiment 18, wherein

the positive control molecule population has binding specificity for each said binding molecule population,

individual members of the positive control molecule population are each bound to a signal generating molecule capable of providing a detectable control signal,

said individual members of the positive control molecule population are bound to the same type of signal generating molecule as said individual members of the capture molecule population, and

said individual members of the positive control molecule population and said individual members of the capture molecule population are each bound to distinct signal generating molecules.

Embodiment 20: The lateral flow device according to any one of embodiments 1 to 15, wherein individual members of each said binding molecule population are bound to a signal generating molecule capable of providing said detectable signal.

Embodiment 21: The lateral flow device according to embodiment 20, wherein

the positive control molecule population comprises known quantities of said multiple analyte populations immobilised within a detection zone of the device in a spatially separated arrangement, and

said positive control molecule population is capable of providing a detectable control signal when bound to said signal generating molecule.

Embodiment 22: The lateral flow device according to any one of embodiments 1 to 15, wherein the multiple analytes are each bound to a signal generating molecule capable of providing said detectable signal.

Embodiment 23: The lateral flow device according to embodiment 22, wherein

the positive control molecule population comprises known quantities of said multiple analyte populations each bound to a signal generating molecule capable of providing a detectable control signal.

Embodiment 24: The lateral flow device according to any one of embodiments 20 to 23 comprising eight or more binding molecule populations selected from: anti-digoxigenin antibodies, anti-TAMRA antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, biotin, and streptavidin.

Embodiment 25: The lateral flow device according to any one of embodiments 1 to 24, wherein the positive control molecule population and/or the capture molecule population is/are present in a conjugate zone of the device.

Embodiment 26: The lateral flow device according to any one of embodiments 1 to 25 comprising any one or more of: a membrane, a sample pad, a conjugate pad, an absorbent pad, an incubation pad, a detection pad, running buffer, and/or plastic housing.

Embodiment 27: The lateral flow device according to any one of embodiments 1 to 26 comprising any one or more of:

a membrane produced from any one or more of nitrocellulose, nylon, polyethersulfone, polyethylene, polyvinylidine difluoride (PVDF), fused silica;

a series of interconnected pads comprising any one or more of: a sample pad for distribution of sample solution to upstream components; a conjugate pad adjacent to the sample pad for controlling release of reactants onto the membrane; an absorbent pad at or close proximity to the base of the lateral flow device for enhancing the capillary driving force and absorbing any unreacted substances; an incubation pad and/or a detection pad adhered to a surface of the membrane for stabilisation of the membrane,

running buffer selected from phosphate-buffered saline (PBS), tris-buffered saline), borate, and buffers comprising blockers including casein, bovine serum albumin (BSA), PVA, and

plastic housing for sealing the device, comprising a sample application inlet and a window above the detection zone.

Embodiment 28: A method for multiplex lateral flow detection of different target analyte populations in a sample, the method comprising:

labelling each target analyte population in the sample with a single type of ligand selected from the group consisting of: digoxigenin, tetramethylrhodamine (TAMRA), dinitrophenyl, Texas Red, cascade blue, streptavidin, biotin, Cy5, dansyl, and fluorescein;

applying the sample to the lateral flow device according to any one of embodiments 1 to 27, and

determining whether one or more individually detectable signals are generated

wherein each individually detectable signal generated is dependent on and indicative of the presence of a specific target analyte population in the sample,

and wherein each said target analyte population for detection in the method is labelled with a different ligand compared to all other target analyte populations, and each said different ligand can contribute to the induction of an individually detectable signal in the lateral flow device.

Embodiment 29: A method for determining an absence of different target analyte populations in a sample by multiplex lateral flow detection, the method comprising:

labelling each target analyte population in the sample with a single type of ligand selected from the group consisting of: digoxigenin, tetramethylrhodamine (TAMRA), Texas Red, dinitrophenyl, cascade blue, streptavidin, biotin, Cy5, dansyl, and fluorescein;

applying the sample to the lateral flow device according to any one of embodiments 1 to 27, and

determining whether one or more individually detectable signals dependent on the presence of a specific target analyte population in the sample are generated,

wherein failure to detect a given signal is indicative of a specific target analyte population being absent in the sample,

and wherein each said target analyte population for detection in the method is labelled with a different ligand compared to all other target analyte populations, and each said different ligand can contribute to the induction of an individually detectable signal in the lateral flow device.

Embodiment 30: The method according to embodiment 28 or embodiment 29, wherein the target analyte populations are nucleic acids, proteins, peptides, lipids, small molecules, or any combination thereof.

Embodiment 31: The method according to any one of embodiments 28 to 30, wherein the target analyte populations are nucleic acids.

Embodiment 32: The method according to embodiment 30 or embodiment 31, wherein the nucleic acids are DNA.

Embodiment 33: The method according to embodiment 31 or embodiment 32, wherein said labelling each target analyte population in the sample comprises: polymerase chain reaction (PCR), isothermal nucleic acid amplification, or a combination thereof.

Embodiment 34: The method according to embodiment 33, wherein the isothermal nucleic acid amplification is selected from any one or more of: LAMP, HDA, NASBA, RPA, RT-PCR or any combination thereof.

Embodiment 35: The method according to any one of embodiments 31 to 33, wherein said labelling each target analyte population in the sample comprises two or more of PCR, RPA, LAMP, HDA, NASBA.

Embodiment 36: The method according to any one of embodiments 28 to 30, wherein at least one of said target analyte populations comprises proteins, peptides, lipids or small molecules, and said labelling of said target analyte comprises use of aptamers and/or antibodies having binding specificity for members of said at least one target analyte population and are each bound to said single type of ligand.

Embodiment 37: The method according to any one of embodiments 28 to 36, wherein:

the target analyte populations are nucleic acids,

said single type of ligand selected from the group consisting of: digoxigenin, Texas Red, dinitrophenyl, cascade blue, biotin, Cy5, dansyl, and fluorescein, is bound to a first terminus of each nucleic acid,

a second terminus of each said nucleic acid is labelled with a ligand selected from the group consisting of: digoxigenin, Texas Red, dinitrophenyl, cascade blue, biotin, Cy5, dansyl, and fluorescein, is bound to a first terminus of each nucleic acid,

the ligand bound to the first terminus is a different type of ligand to that which is bound to the second terminus, and

the ligand bound to the second terminus is the same in nucleic acids of all target analyte populations.

Embodiment 38: The method according to any one of embodiments 28 to 37, wherein the detectable signal is a colourimetric signal including a signal generated from enzymes or enzyme substrates, beads, particles, fluorescent dyes, nanomaterials (e.g. latex beads or colloidal gold particles, carbon particles, magnetic particles, paramagnetic particles, quantum dots, up-converting phosphorus, nano microspheres, nano-tubes, chelate-loaded silica, europium), liposomes, and fluorescent immunoliposomes.

Embodiment 39: A method for producing a lateral flow device, the method comprising depositing at least three binding molecule populations in a detection zone of the lateral flow device, wherein each said binding molecule population:

has binding specificity for a different target ligand;

has less than: 10%, 5%, 4%, 3%, 2%, or 1%, cross-reactivity with a target ligand for which any other said binding molecule population has binding specificity;

is immobilised within a detection zone of the device, and spatially separated from all other detection molecule populations in the detection zone; and

is capable of contributing to the formation of a signalling complex capable of providing a detectable signal only in the presence of a target analyte, wherein the signal complex comprises a member of any one of said binding molecule populations bound to said target ligand for which the member has binding specificity, which is in turn bound to said target analyte.

Embodiment 40: The method according to embodiment 39, wherein each said binding molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin and biotin.

Embodiment 41: The lateral flow device according to embodiment 39 or embodiment 40 wherein

the binding molecule that exhibits the lowest level of sensitivity in the presence of its target analyte is positioned closer to a sample application zone of the device compared to any other binding molecule population immobilised in the detection zone.

Embodiment 42: The lateral flow device according to any one of embodiments 39 to 41, wherein

the binding molecule that exhibits the highest level of sensitivity in the presence of its target analyte is positioned furthest from a sample application zone of the device compared to any other analyte immobilised in the detection zone.

Embodiment 43: The method according to any one of embodiments 39 to 42, wherein the spatially separated arrangement is any one or more of: non-linear, a non-linear dot or line format and/or a dot matrix format.

Embodiment 44: The method according to any one of embodiments 39 to 43 comprising depositing seven different detection molecule populations, wherein

each is immobilised within the detection zone of the device in a dot matrix format, wherein

each of three of the binding molecule populations are represented in the dot matrix by two dots per population which are equidistant or substantially equidistant from a sample application zone of the device, and

each of four of the binding molecule populations are represented in the dot matrix by one dot per population which are at different distances from a sample application zone of the device, and

all said dots are collectively arranged in a pattern forming the digit eight.

Embodiment 45: The method according to any one of embodiments 39 to 44, wherein the three binding molecule populations are a combination shown in Table 3.

Embodiment 46: The method according to any one of embodiments 39 to 45 comprising depositing four binding molecule populations in the detection zone, wherein the four binding molecule populations are a combination shown in Table 4.

Embodiment 47: The method according to any one of embodiments 39 to 46 comprising depositing five binding molecule populations in the detection zone, wherein the five binding molecule populations are a combination shown in Table 5.

Embodiment 48: The lateral flow device according to any one of embodiments 39 to 47 comprising depositing six binding molecule populations in the detection zone, wherein the six binding molecule populations are a combination shown in Table 6.

Embodiment 49: The method according to any one of embodiments 39 to 48, comprising depositing seven binding molecule populations in the detection zone, wherein the seven binding molecule populations are a combination shown in Table 7.

Embodiment 50: The method according to embodiment 49, wherein the seven detection molecule populations are anti-digoxigenin antibodies, anti-TAMRA antibodies; anti-Texas Red antibodies, anti-dinitrophenyl antibodies, anti-Cascade Blue antibodies, either one of streptavidin or anti-biotin antibodies, and either one of anti-Dansyl antibodies or anti-Cy5 antibodies.

Embodiment 51: The method according to any one of embodiments 39 to 50, further comprising including a positive control molecule population in the device.

Embodiment 52: The method according to any one of embodiments 39 to 51, further comprising including a capture molecule population in the device having binding specificity for a capture ligand, wherein,

the capture molecule population and each said binding molecule population have binding specificity for different target ligands;

the capture molecule population has less than: 10%, 5%, 4%, 3%, 2%, or 1%, cross-reactivity with a target ligand for which any other said binding molecule population has binding specificity;

individual members of the capture molecule population are each bound to a signal generating molecule capable of providing said detectable signal; and

said signal complex comprises a member of the capture molecule population bound to said capture ligand which is in turn bound to said target analyte.

Embodiment 53: The method according to embodiment 52, wherein the capture molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin, and biotin.

Embodiment 54: The method according to embodiment 52 or embodiment 53, wherein

the positive control molecule population has binding specificity for each said binding molecule population,

individual members of the positive control molecule population are each bound to a signal generating molecule capable of providing a detectable control signal,

said individual members of the positive control molecule population are bound to the same type of signal generating molecule as said individual members of the capture molecule population, and

said individual members of the positive control molecule population and said individual members of the capture molecule population are each bound to distinct signal generating molecules.

Embodiment 55: The method according to any one of embodiments 39 to 54 comprising incorporating into the device any one or more of: a membrane, a sample pad, a conjugate pad, an absorbent pad, an incubation pad, a detection pad, running buffer, and/or plastic housing.

Embodiment 56: The method according to any one of embodiments 39 to 55 comprising incorporating into the device any one or more of:

a membrane produced from any one or more of nitrocellulose, nylon, polyethersulfone, polyethylene, polyvinylidine difluoride (PVDF), fused silica;

a series of interconnected pads comprising any one or more of: a sample pad for distribution of sample solution to upstream components; a conjugate pad adjacent to the sample pad for controlling release of reactants onto the membrane; an absorbent pad at or close proximity to the base of the lateral flow device for enhancing the capillary driving force and absorbing any unreacted substances; an incubation pad and/or a detection pad adhered to a surface of the membrane for stabilisation of the membrane,

running buffer selected from phosphate-buffered saline (PBS) tris-buffered saline), borate, and buffers comprising blockers including casein, bovine serum albumin (BSA), PVA, and

plastic housing for sealing the device, comprising a sample application inlet and a window above the detection zone.

Embodiment 57: A lateral flow device obtained or obtainable by the method of any one of embodiments 39 to 56.

### BRIEF DESCRIPTION OF THE DRAWINGS

Preferred embodiments of the present invention will now be described, by way of example only, with reference to the accompanying figures wherein:

FIG. 1 depicts a representative sandwich immunoassay (FIG. 1a) that can be used in lateral flow devices (FIG. 1b) of the present invention;

FIG. 2 shows single-plex lateral flow detection results for twelve antigen-antibody pairs. FIG. 2A demonstrates that all twelve antigen-antibody pairs were effective as detection entities in the single-plex LFDs. FIGS. 2B-D are a series of graphs each indicative of the lower detection limits of various antigen-antibody pairs in a detection assay according to the present invention. Colour intensity (quantitated using ImageJ software) is plotted against DNA concentration with data points and error bars indicating the average and standard deviation of 4 individual tests. The solid black line represents the cut-off used to determine the lowest concentration at which a signal could still be detected (defined as three standard deviations above the average negative values);

FIG. 3 depicts a multiplex LFD array according to the present invention incorporating twelve antigen-detection antibody pairs (FIG. 3A), and provides specificity test results for each antigen-detection antibody pair in the array (FIG. 3B). The black arrow denotes the correct test dot while the red arrow denotes the incorrect test dot. The assay was independently performed three times with similar results and a representative photograph from one test is shown;

FIG. 4 depicts a hepta-plex LFD array according to the present invention incorporating seven antigen-detection antibody pairs (FIG. 4A), and shows test results for each antigen-detection antibody pair the assay (FIG. 4B);

FIG. 5 shows a hepta-plex LFD array according to the present invention. FIG. 5A indicates positioning of the detection antibodies that form the 7-segments of the display. FIG. 5B depicts test results upon addition of labelled analyte signature mixtures to the LFD array;

FIG. 6 shows another hepta-plex LFD array according to the present invention. FIG. 6A indicates positioning of the detection antibodies that form the 7-segments of the display. FIG. 6B depicts test results upon addition of labelled analyte signature mixtures to the LFD array;

FIG. 7 relates to an RPA and single-plex lateral flow sandwich assay according to the present invention;

FIG. 8 shows results of single-plex RPA in combination with multiplexed lateral flow detection according to the present invention; and

FIG. 9 shows results of multiplex RPA in combination with multiplex lateral flow detection according to the present invention.

### DEFINITIONS

As used in this application, the singular form “a”, “an” and “the” include plural references unless the context clearly dictates otherwise. For example, the phrase “an antibody” also includes a plurality of antibodies.

As used herein, the term “comprising” means “including.” Variations of the word “comprising”, such as “comprise” and “comprises,” have correspondingly varied meanings. Thus, for example, an LFD “comprising” three binding molecule populations may consist exclusively of three binding molecule populations or may include one or more additional binding molecule populations (e.g. four, five six, or seven binding molecule populations).

As used herein, the terms “antibody” and “antibodies” include IgG (including IgG1, IgG2, IgG3, and IgG4), IgA (including IgAl and IgA2), IgD, IgE, or IgM, and IgY, whole antibodies, including single-chain whole antibodies, and antigen-binding fragments thereof. Antigen-binding antibody fragments include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, multispecific, humanized, and human monoclonal and polyclonal antibodies which specifically bind the biological molecule.

As used herein the term “nucleic acid” refers to a molecule comprising one or more nucleic acid subunits which may be selected from adenosine (A), cytosine (C), guanine (G), thymine (T), uracil (U), and known analogues thereof that can hybridise to nucleic acids in a manner similar to naturally occurring nucleotides. In some examples, the nucleic acid may comprise or consist of a deoxyribonucleic acid (DNA), a ribonucleic acid (RNA), or complementary deoxyribonucleic acid (DNA). The nucleic acid may be a polymer of nucleic acids in either single- or double-stranded form.

As used herein, the term “polynucleotide” refers to a single- or double-stranded polymer of deoxyribonucleotide bases, ribonucleotide bases, known analogues or natural nucleotides, or mixtures thereof.

As used herein, the terms “binds specifically”, and “binding specificity” in reference to an antibody, antibody variant, antibody derivative, antigen binding fragment, and the like refers to its capacity to bind to a given target ligand preferentially over other non-target ligands. For example, if the antibody, antibody variant, antibody derivative, or antigen binding fragment (“molecule A”) is capable of “binding specifically” to a given target ligand (“molecule B”), molecule A has the capacity to discriminate between molecule B and any other number of potential alternative binding partners. Accordingly, when exposed to a plurality of different but equally accessible molecules as potential binding partners, molecule A will selectively bind to molecule B and other alternative potential binding partners will remain substantially unbound by molecule A. In general, molecule A will preferentially bind to molecule B at least 10-fold, preferably 50-fold, more preferably 100-fold, and most preferably greater than 100-fold more frequently than other potential binding partners. Molecule A may be capable of binding to molecules that are not molecule B at a weak, yet detectable level. This is commonly known as background binding and is readily discernible from molecule B-specific binding, for example, by use of an appropriate control.

As used herein, the term “binding molecule” refers to a molecule that binds specifically to given target molecule.

As used herein, the term “binding molecule population” refers to a discrete population of molecules, each of which has binding specificity for the same type of target ligand.

As used herein, the term “analyte” encompasses any compound, molecule, or other substance of interest to be detected by the methods of the present invention.

Any description of prior art documents herein, or statements herein derived from or based on those documents, is not an admission that the documents or derived statements are part of the common general knowledge of the relevant art.

For the purposes of description all documents referred to herein are hereby incorporated by reference in their entirety unless otherwise stated.

### DETAILED DESCRIPTION

In general, existing multiplex LFDs are only capable of detecting a limited number of analytes. Strategies adopted to increase the number of analytes detectable have suffered from problems including higher reagent consumption, increased dimension size, and reduced sensitivity.

The present inventors have devised strategies for improving multiplex detection of analytes in LFDs. In addressing the problems in the prior art, it was necessary for the inventors to identify a means of increasing the number of analytes detectable by multiplex LFDs in consideration of concerns such as minimising reagent consumption, maintaining the sensitivity/specificity of analyte detection, and preserving the compactness and ease of use of the device.

The LFDs described herein provide a unique system of generic application, in which individual target analytes are labelled with different ligands each of which is a binding partner for a specific type of antibody. Binding of a given target analyte via its associated ligand to the antibody provides or contributes to a signalling complex within a detection zone of the device. The signalling complexes so formed are arranged in a manner which facilitates the creation of signature patterns within the detection zone. The combinations of ligand/antibody pairs forming the signature patterns can be selected on the basis of minimising cross-reactivity between pairs, and may be ordered moving outward from the sample inlet on the basis of lowest to highest sensitivity. As a result, the signature patterns significantly increase the number of entities detectable by the device while minimising space requirements. This means that the sample need not flow excessive distances in the device thus improving reagent consumption and sensitivity of detection while maintaining the compactness of the device.

The following detailed description conveys exemplary embodiments of the present invention in sufficient detail to enable those of ordinary skill in the art to practice the present invention. Features or limitations of the various embodiments described do not necessarily limit other embodiments of the present invention or the present invention as a whole. Hence, the following detailed description does not limit the scope of the present invention, which is defined only by the claims.

Detection Complexes

The devices and methods according to the present invention employ combinations of different binding molecule/ligand pairs for multiplex analyte detection. A target analyte to be detected may be labelled with at least one type of ligand, and a binding molecule with binding specificity for the ligand can then hybridise to it providing an analyte-ligand-binding molecule complex.

For exemplary purposes only, an LFD designed to detect seven different types of analytes (analytes A, B, C, D, E, F, G) may incorporate seven different types of ligands (ligands 1, 2, 3, 4, 5, 6 and 7). Each analyte could be labelled directly or indirectly with a different type of ligand (e.g. analyte A/ligandl; analyte B/ligand 2; analyte C/ligand 3; analyte D/ligand 4; analyte E/ligand 5; analyte F/ligand 6; analyte G/ligand 7). The LFD may also utilise seven different types of binding molecules (e.g. antibodies), each type of binding molecule having binding specificity for only one ligand type. Accordingly, analyte-ligand-binding molecule complexes may be formed, each indicative of the presence of a specific type of analyte in a test sample.

Depending on the format of the LFD assay, the binding molecules may be immobilised in a detection zone of the LFD to capture analyte-ligand combinations applied in solution to the detection zone. Alternatively, the analyte-ligand combination may be immobilised in the detection zone and binding molecules with binding specificity for the ligand applied to form analyte-ligand-binding molecule complexes. In either scenario, the immobilised entity can be spatially separated from other different types of immobilised entities such that discrete populations of immobilised entities exist that are of different types.

The analyte-ligand-binding molecule complexes may produce or contribute to the production of a detectable signal indicative of the presence of the analyte. Again depending on the format of the LFD assay, the binding molecule or the analyte may be directly bound to a signalling molecule.

Alternatively, the analyte may be labelled with first and second ligand types (e.g. any one of ligands 1, 2, 3, 4, 5, 6 or 7 as mentioned above) with the caveat that the first ligand is a different type of ligand to the second ligand bound to the analyte. Accordingly, the binding molecule that may bind with the first ligand can be of a different type to a binding molecule that can bind to the second ligand. In LFD assays in which analytes are each labelled with two different types of ligands, it may be desirable to ensure that all analytes to be detected are labelled with the same type of second ligand. First and second antibody types having binding specificity for the first and second ligand, respectively, can form a complex as follows: 1^{st} binding molecule—1^{st} ligand—analyte—2^{nd} ligand—2^{nd} binding molecule. The first binding molecule may be immobilised to the detection zone of the LFD. The second binding molecule may be, or may be bound to, a signalling molecule capable of providing a detectable signal indicative of the presence of the analyte. Signalling molecules bound to second binding molecules complexed with different analyte types may be of the same type, or, of a different type.

Binding Molecules

Binding molecules according to the present invention may include any molecules that bind specifically to a given target analyte via a ligand directly or indirectly bound to the analyte. The degree of cross-reactivity (i.e. non-specific binding) between different types of binding molecules and different types of ligands present in the LFD is preferably minimal or non-existent. By way of non-limiting example, the binding molecules may be antibodies (including antigen-binding fragments or derivatives thereof) or aptamers (e.g. nucleic acid aptamers, peptide aptamers, and the like).

In some embodiments of the present invention, the LFD may comprise any one or more of the binding molecule/ligand pairs shown in Table 1.

TABLE ONE
binding molecule/ligand pairs

| Binding Molecule | Ligand |
|---|---|
| Anti - Digoxigenin (DIG)^{#} | Digoxigenin* |
| Anti - Texas Red^{#} | Texas Red* |
| (sulforhodamine 101 acid chloride) | |
| Anti - Cascade Blue^{#} | Cascade Blue* |
| (N-[4-[[4-(diethylamino)phenyl][4-(ethylamino)-1- | |
| naphthalenyl]methylene]-2,5-cyclohexadien-1-ylidene]- | |
| N-ethyl-Ethanaminium, molybdatetungstate phosphate) | |
| Anti -Tetramethylrhodamine (TAMRA)^{#} | TAMRA* |
| (Carboxytetramethylrhodamine) | |
| Anti - Dinitrophenol^{#} | Dinitrophenol* |
| Anti - Biotin antibody^{#} | Biotin* |
| Anti - Streptavidin antibody^{#} | Streptavidin* |
| Anti - Cyanine 5 (Cy5) antibody^{#} | Cyanine 5* |
| (1,1′-bis(3-hydroxypropyl)-3,3,3′,3′- | |
| tetramethylindodicarbocyanine) | |
| Anti - Dansyl antibody^{#} | Dansyl* |
| ([5-(dimethylamino)naphthalene-1-sulfonyl]) | |
| Biotin* | Streptavidin* |
| Streptavidin* | Biotin* |

Table 1 key:
^{#}includes antigen-binding fragments and derivatives thereof
*includes fragments and derivatives thereof

The skilled addressee will understand that a “derivative” of a ligand referred to herein such as, for example, derivatives of any one or more of digoxigenin, Texas Red, Cascade Blue, tetramethylrhodamine (TAMRA), dinitrophenol, biotin, streptavidin, Cyanine 5, and Dansyl, can be prepared from the parent ligand (or de novo) using standard techniques. Although the derivative may differ in chemical structure from the parent ligand, the parent ligand or a portion thereof may be a component of the derivative. Derivatives of digoxigenin, Texas Red, Cascade Blue, tetramethylrhodamine (TAMRA), dinitrophenol, biotin, and streptavidin, are well known to those of ordinary skill in the art, as are methods for their production.

The ligand component of each binding molecule/ligand pair may be directly or indirectly bound to an analyte. Ligands of different binding molecule/ligand pairs may be directly or indirectly bound to the same type of analyte or to a different type of analyte. The binding molecule component of each binding molecule/ligand pair may be immobilised in a detection zone of the LFD, or alternatively may be bound to a signalling molecule.

In some embodiments of the present invention, the LFD may comprise a combination of two binding molecule/ligand pairs shown in Table 2 below.

TABLE TWO
combinations of two binding molecule/ligand pairs

| | | | | | | | | |
|---|---|---|---|---|---|---|---|---|
| 1 + 2 | 2 + 3 | 3 + 4 | 4 + 5 | 5 + 6 | 6 + 7 | 7 + 8 | 8 + 9 | 9 + 10 |
| 1 + 3 | 2 + 4 | 3 + 5 | 4 + 6 | 5 + 7 | 6 + 8 | 7 + 9 | 8 + 10 | |
| 1 + 4 | 2 + 5 | 3 + 6 | 4 + 7 | 5 + 8 | 6 + 9 | 7 + 10 | | |
| 1 + 5 | 2 + 6 | 3 + 7 | 4 + 8 | 5 + 9 | 6 + 10 | | | |
| 1 + 6 | 2 + 7 | 3 + 8 | 4 + 9 | 5 + 10 | | | | |
| 1 + 7 | 2 + 8 | 3 + 9 | 4 + 10 | | | | | |
| 1 + 8 | 2 + 9 | 3 + 10 | | | | | | |
| 1 + 9 | 2 + 10 | | | | | | | |
| 1 + 10 | | | | | | | | |

Table Two key -
1: Anti-DIG antibody^{#}/DIG*
2: Anti-Texas Red antibody^{#}/Texas Red*
3: Anti-Cascade Blue antibody^{#}/Cascade Blue *
4: Anti-TAMRA antibody^{#}/TAMRA *
5: Anti-Dinitrophenol antibody^{#}/Dinitrophenol*
6: Anti-Cy5 antibody^{#}/Cy5*
7: Anti-Dansyl antibody^{#}/Dansyl*
8: Anti-Streptavidin antibody^{#}/Streptavidin*
9: Anti-Biotin antibody^{#}/Biotin*
10: Strepatividin^{#}/Biotin*
^{#}includes antigen-binding fragments and derivatives thereof
*includes fragments and derivatives thereof

The binding molecule of either or both pairs may be immobilised in a detection zone of the LFD. Alternatively, the binding molecule of either or both pairs may be directly or indirectly bound to a signalling molecule.

In some embodiments of the present invention, the LFD may comprise a combination of three binding molecule/ligand pairs shown in Table 3 below.

TABLE THREE
combinations of three binding molecule/ligand pairs

| | | | | | | | |
|---|---|---|---|---|---|---|---|
| 1 + 2 + 3 | 2 + 3 + 4 | 3 + 4 + 5 | 4 + 5 + 6 | 5 + 6 + 7 | 6 + 7 + 8 | 7 + 8 + 9 | 8 + 9 + 10 |
| 1 + 2 + 4 | 2 + 3 + 5 | 3 + 4 + 6 | 4 + 5 + 7 | 5 + 6 + 8 | 6 + 7 + 9 | 7 + 8 + 10 | |
| 1 + 2 + 5 | 2 + 3 + 6 | 3 + 4 + 7 | 4 + 5 + 8 | 5 + 6 + 9 | 6 + 7 + 10 | 7 + 8 + 9 | |
| 1 + 2 + 6 | 2 + 3 + 7 | 3 + 4 + 8 | 4 + 5 + 9 | 5 + 6 + 10 | 6 + 8 + 9 | 7 + 8 + 10 | |
| 1 + 2 + 7 | 2 + 3 + 8 | 3 + 4 + 9 | 4 + 5 + 10 | 5 + 7 + 8 | 6 + 8 + 10 | 7 + 9 + 10 | |
| 1 + 2 + 8 | 2 + 3 + 9 | 3 + 4 + 10 | 4 + 6 + 7 | 5 + 7 + 9 | 6 + 9 + 10 | | |
| 1 + 2 + 9 | 2 + 3 + 10 | 3 + 5 + 6 | 4 + 6 + 8 | 5 + 7 + 10 | | | |
| 1 + 2 + 10 | 2 + 4 + 5 | 3 + 5 + 7 | 4 + 6 + 9 | 5 + 8 + 9 | | | |
| 1 + 3 + 4 | 2 + 4 + 6 | 3 + 6 + 7 | 4 + 6 + 10 | 5 + 8 + 10 | | | |
| 1 + 3 + 5 | 2 + 4 + 7 | 3 + 6 + 8 | 4 + 7 + 8 | 5 + 9 + 10 | | | |
| 1 + 3 + 6 | 2 + 5 + 6 | 3 + 6 + 9 | 4 + 7 + 9 | | | | |
| 1 + 3 + 7 | 2 + 5 + 7 | 3 + 6 + 10 | 4 + 7 + 10 | | | | |
| 1 + 3 + 8 | 2 + 5 + 8 | 3 + 7 + 8 | 4 + 8 + 9 | | | | |
| 1 + 3 + 9 | 2 + 5 + 9 | 3 + 7 + 9 | 4 + 8 + 10 | | | | |
| 1 + 3 + 10 | 2 + 5 + 10 | 3 + 7 + 10 | 4 + 9 + 10 | | | | |
| 1 + 4 + 5 | 2 + 6 + 7 | 3 + 8 + 9 | | | | | |
| 1 + 4 + 6 | 2 + 6 + 8 | 3 + 8 + 10 | | | | | |
| 1 + 4 + 7 | 2 + 6 + 9 | 3 + 9 + 10 | | | | | |
| 1 + 4 + 8 | 2 + 6 + 10 | | | | | | |
| 1 + 4 + 9 | 2 + 7 + 8 | | | | | | |
| 1 + 4 + 10 | 2 + 7 + 9 | | | | | | |
| 1 + 5 + 6 | 2 + 7 + 10 | | | | | | |
| 1 + 5 + 7 | 2 + 8 + 9 | | | | | | |
| 1 + 5 + 8 | 2 + 8 + 10 | | | | | | |
| 1 + 5 + 9 | 2 + 9 + 10 | | | | | | |
| 1 + 5 + 10 | | | | | | | |
| 1 + 6 + 7 | | | | | | | |
| 1 + 6 + 8 | | | | | | | |
| 1 + 6 + 9 | | | | | | | |
| 1 + 6 + 10 | | | | | | | |
| 1 + 7 + 8 | | | | | | | |
| 1 + 7 + 9 | | | | | | | |
| 1 + 7 + 10 | | | | | | | |
| 1 + 8 + 9 | | | | | | | |
| 1 + 8 + 10 | | | | | | | |
| 1 + 9 + 10 | | | | | | | |

Table Three key -
1: Anti-DIG antibody^{#}/DIG*
2: Anti-Texas Red antibody^{#}/Texas Red*
3: Anti-Cascade Blue antibody^{#}/Cascade Blue *
4: Anti-TAMRA antibody^{#}/TAMRA *
5: Anti-Dinitrophenol antibody^{#}/Dinitrophenol*
6: Anti-Cy5 antibody^{#}/Cy5*
7: Anti-Dansyl antibody^{#}/Dansyl*
8: Anti-Streptavidin antibody^{#}/Streptavidin*
9: Anti-Biotin antibody^{#}/Biotin*
10: Strepatividin^{#}/Biotin*
^{#}includes antigen-binding fragments and derivatives thereof
*includes fragments and derivatives thereof

The binding molecule of any one, any two, or all three pairs may be immobilised in a detection zone of the LFD. Alternatively, the binding molecule of any one, any two, or all three pairs may be directly or indirectly bound to a signalling molecule.

In some embodiments of the present invention, the LFD may comprise a combination of four binding molecule/ligand pairs shown in Table 4 below.

TABLE FOUR
combinations of four binding molecule/ligand pairs

| | | | | | |
|---|---|---|---|---|---|
| 1 + 2 + 3 + 4 | 2 + 3 + 4 + 5 | 3 + 4 + 5 + 6 | 4 + 5 + 6 + 7 | 5 + 6 + 7 + 8 | 6 + 7 + 8 + 9 |
| 1 + 2 + 3 + 5 | 2 + 3 + 4 + 6 | 3 + 4 + 5 + 7 | 4 + 5 + 6 + 8 | 5 + 6 + 7 + 9 | 6 + 7 + 8 + 10 |
| 1 + 2 + 3 + 6 | 2 + 3 + 4 + 7 | 3 + 4 + 5 + 8 | 4 + 5 + 6 + 9 | 5 + 6 + 7 + 10 | 6 + 8 + 9 + 10 |
| 1 + 2 + 3 + 7 | 2 + 3 + 5 + 6 | 3 + 4 + 5 + 9 | 4 + 5 + 6 + 10 | 5 + 6 + 8 + 9 | |
| 1 + 2 + 3 + 8 | 2 + 3 + 5 + 7 | 3 + 4 + 5 + 10 | 4 + 5 + 7 + 8 | 5 + 6 + 8 + 10 | |
| 1 + 2 + 3 + 9 | 2 + 3 + 5 + 8 | 3 + 4 + 6 + 7 | 4 + 5 + 7 + 9 | 5 + 6 + 9 + 10 | |
| 1 + 2 + 3 + 10 | 2 + 3 + 5 + 9 | 3 + 4 + 6 + 8 | 4 + 5 + 7 + 10 | 5 + 7 + 8 + 9 | |
| 1 + 2 + 4 + 5 | 2 + 3 + 5 + 10 | 3 + 4 + 6 + 9 | 4 + 5 + 8 + 9 | 5 + 7 + 8 + 10 | |
| 1 + 2 + 4 + 6 | 2 + 3 + 6 + 7 | 3 + 4 + 6 + 10 | 4 + 5 + 8 + 10 | 5 + 7 + 9 + 10 | |
| 1 + 2 + 4 + 7 | 2 + 3 + 6 + 8 | 3 + 4 + 7 + 8 | 4 + 5 + 9 + 10 | 5 + 8 + 9 + 10 | |
| 1 + 2 + 4 + 8 | 2 + 3 + 6 + 9 | 3 + 4 + 7 + 9 | 4 + 6 + 7 + 8 | | |
| 1 + 2 + 4 + 9 | 2 + 3 + 6 + 10 | 3 + 4 + 7 + 10 | 4 + 6 + 7 + 9 | | |
| 1 + 2 + 4 + 10 | 2 + 3 + 7 + 8 | 3 + 4 + 8 + 9 | 4 + 6 + 7 + 10 | | |
| 1 + 2 + 5 + 6 | 2 + 3 + 7 + 9 | 3 + 4 + 8 + 10 | 4 + 6 + 8 + 9 | | |
| 1 + 2 + 5 + 7 | 2 + 3 + 7 + 10 | 3 + 4 + 9 + 10 | 4 + 6 + 8 + 10 | | |
| 1 + 2 + 5 + 8 | 2 + 3 + 8 + 9 | 3 + 5 + 6 + 7 | 4 + 6 + 9 + 10 | | |
| 1 + 2 + 5 + 9 | 2 + 3 + 8 + 10 | 3 + 5 + 6 + 8 | 4 + 7 + 8 + 9 | | |
| 1 + 2 + 5 + 10 | 2 + 3 + 9 + 10 | 3 + 5 + 6 + 9 | 4 + 7 + 8 + 10 | | |
| 1 + 2 + 6 + 7 | 2 + 4 + 5 + 6 | 3 + 5 + 6 + 10 | 4 + 7 + 9 + 10 | | |
| 1 + 2 + 6 + 8 | 2 + 4 + 5 + 7 | 3 + 5 + 7 + 8 | 4 + 8 + 9 + 10 | | |
| 1 + 2 + 6 + 9 | 2 + 4 + 5 + 8 | 3 + 5 + 7 + 9 | | | |
| 1 + 2 + 6 + 10 | 2 + 4 + 5 + 9 | 3 + 5 + 7 + 10 | | | |
| 1 + 2 + 7 + 8 | 2 + 4 + 5 + 10 | 3 + 5 + 7 + 8 | | | |
| 1 + 2 + 7 + 9 | 2 + 4 + 6 + 7 | 3 + 5 + 7 + 9 | | | |
| 1 + 2 + 7 + 10 | 2 + 4 + 6 + 8 | 3 + 5 + 7 + 10 | | | |
| 1 + 2 + 8 + 9 | 2 + 4 + 6 + 9 | 3 + 5 + 8 + 9 | | | |
| 1 + 2 + 8 + 10 | 2 + 4 + 6 + 10 | 3 + 5 + 8 + 10 | | | |
| 1 + 2 + 9 + 10 | 2 + 4 + 6 + 7 | 3 + 5 + 9 + 10 | | | |
| 1 + 3 + 4 + 5 | 2 + 4 + 6 + 8 | 3 + 6 + 7 + 8 | | | |
| 1 + 3 + 4 + 6 | 2 + 4 + 6 + 9 | 3 + 6 + 7 + 9 | | | |
| 1 + 3 + 4 + 7 | 2 + 4 + 6 + 10 | 3 + 6 + 7 + 10 | | | |
| 1 + 3 + 4 + 8 | 2 + 4 + 7 + 8 | 3 + 7 + 8 + 9 | | | |
| 1 + 3 + 4 + 9 | 2 + 4 + 7 + 9 | 3 + 7 + 8 + 10 | | | |
| 1 + 3 + 4 + 10 | 2 + 4 + 7 + 10 | 3 + 7 + 9 + 10 | | | |
| 1 + 3 + 5 + 6 | 2 + 4 + 8 + 9 | 3 + 8 + 9 + 10 | | | |
| 1 + 3 + 5 + 7 | 2 + 4 + 8 + 10 | | | | |
| 1 + 3 + 5 + 8 | 2 + 4 + 9 + 10 | | | | |
| 1 + 3 + 5 + 9 | 2 + 5 + 6 + 7 | | | | |
| 1 + 3 + 5 + 10 | 2 + 5 + 6 + 8 | | | | |
| 1 + 3 + 6 + 7 | 2 + 5 + 6 + 9 | | | | |
| 1 + 3 + 6 + 8 | 2 + 5 + 6 + 10 | | | | |
| 1 + 3 + 6 + 9 | 2 + 5 + 7 + 8 | | | | |
| 1 + 3 + 6 + 10 | 2 + 5 + 7 + 9 | | | | |
| 1 + 3 + 7 + 8 | 2 + 5 + 7 + 10 | | | | |
| 1 + 3 + 7 + 9 | 2 + 5 + 7 + 8 | | | | |
| 1 + 3 + 7 + 10 | 2 + 5 + 7 + 9 | | | | |
| 1 + 3 + 8 + 9 | 2 + 5 + 7 + 10 | | | | |
| 1 + 3 + 8 + 10 | 2 + 5 + 8 + 9 | | | | |
| 1 + 3 + 9 + 10 | 2 + 5 + 8 + 10 | | | | |
| 1 + 4 + 5 + 6 | 2 + 5 + 9 + 10 | | | | |
| 1 + 4 + 5 + 7 | 2 + 6 + 7 + 8 | | | | |
| 1 + 4 + 5 + 8 | 2 + 6 + 7 + 9 | | | | |
| 1 + 4 + 5 + 9 | 2 + 6 + 7 + 10 | | | | |
| 1 + 4 + 5 + 10 | 2 + 6 + 8 + 9 | | | | |
| 1 + 4 + 6 + 7 | 2 + 6 + 8 + 10 | | | | |
| 1 + 4 + 6 + 8 | 2 + 6 + 9 + 10 | | | | |
| 1 + 4 + 6 + 9 | 2 + 7 + 8 + 9 | | | | |
| 1 + 4 + 6 + 10 | 2 + 7 + 8 + 10 | | | | |
| 1 + 4 + 7 + 8 | 2 + 7 + 9 + 10 | | | | |
| 1 + 4 + 7 + 9 | 2 + 8 + 9 + 10 | | | | |
| 1 + 4 + 7 + 10 | | | | | |
| 1 + 4 + 8 + 9 | | | | | |
| 1 + 5 + 6 + 7 | | | | | |
| 1 + 5 + 6 + 8 | | | | | |
| 1 + 5 + 6 + 9 | | | | | |
| 1 + 5 + 6 + 10 | | | | | |
| 1 + 5 + 7 + 8 | | | | | |
| 1 + 5 + 7 + 9 | | | | | |
| 1 + 5 + 7 + 10 | | | | | |
| 1 + 5 + 8 + 9 | | | | | |
| 1 + 6 + 7 + 8 | | | | | |
| 1 + 6 + 7 + 9 | | | | | |
| 1 + 6 + 7 + 10 | | | | | |
| 1 + 6 + 8 + 9 | | | | | |
| 1 + 6 + 8 + 10 | | | | | |
| 1 + 6 + 9 + 10 | | | | | |
| 1 + 7 + 8 + 9 | | | | | |
| 1 + 7 + 8 + 10 | | | | | |
| 1 + 7 + 9 + 10 | | | | | |
| 1 + 8 + 9 + 10 | | | | | |

Table Four key -
1: Anti-DIG antibody#/DIG*
2: Anti-Texas Red antibody#/Texas Red*
3: Anti-Cascade Blue antibody#/Cascade Blue*
4: Anti-TAMRA antibody#/TAMRA*
5: Anti-Dinitrophenol antibody#/Dinitrophenol*
6: Anti-Cy5 antibody#/Cy5*
7: Anti-Dansyl antibody#/Dansyl*
8: Anti-Streptavidin antibody#/Streptavidin*
9: Anti-Biotin antibody#/Biotin*
10: Strepatividin#/Biotin*
#includes antigen-binding fragments and derivatives thereof
*includes fragments and derivatives thereof

The binding molecule of any one, any two, any three, or all four pairs may be immobilised in a detection zone of the LFD. Alternatively, the binding molecule of any one, any two, any three, or all four pairs may be directly or indirectly bound to a signalling molecule.

In some embodiments of the present invention, the LFD may comprise a combination of five binding molecule/ligand pairs shown in Table 5 below.

TABLE FIVE
combinations of five binding molecule/ligand pairs

| | | | | |
|---|---|---|---|---|
| 1 + 2 + 3 + 4 + 5 | 2 + 3 + 4 + 5 + 6 | 3 + 4 + 5 + 6 + 7 | 4 + 5 + 6 + 7 + 8 | 5 + 6 + 7 + 8 + 9 |
| 1 + 2 + 3 + 4 + 6 | 2 + 3 + 4 + 5 + 7 | 3 + 4 + 5 + 6 + 8 | 4 + 5 + 6 + 7 + 9 | 5 + 6 + 7 + 8 + 10 |
| 1 + 2 + 3 + 4 + 7 | 2 + 3 + 4 + 5 + 8 | 3 + 4 + 5 + 6 + 9 | 4 + 5 + 6 + 7 + 10 | 5 + 6 + 7 + 9 + 10 |
| 1 + 2 + 3 + 4 + 8 | 2 + 3 + 4 + 5 + 9 | 3 + 4 + 5 + 6 + 10 | 4 + 5 + 6 + 8 + 9 | 5 + 6 + 8 + 9 + 10 |
| 1 + 2 + 3 + 4 + 9 | 2 + 3 + 4 + 5 + 10 | 3 + 4 + 5 + 7 + 8 | 4 + 5 + 6 + 8 + 10 | 5 + 7 + 8 + 9 + 10 |
| 1 + 2 + 3 + 4 + 10 | 2 + 3 + 4 + 6 + 7 | 3 + 4 + 5 + 7 + 9 | 4 + 5 + 6 + 9 + 10 | |
| 1 + 2 + 3 + 5 + 6 | 2 + 3 + 4 + 6 + 8 | 3 + 4 + 5 + 7 + 10 | 4 + 5 + 7 + 8 + 9 | 6 + 7 + 8 + 9 + 10 |
| 1 + 2 + 3 + 5 + 7 | 2 + 3 + 4 + 6 + 9 | 3 + 4 + 5 + 8 + 9 | 4 + 5 + 7 + 8 + 10 | |
| 1 + 2 + 3 + 5 + 8 | 2 + 3 + 4 + 6 + 10 | 3 + 4 + 5 + 8 + 10 | 4 + 5 + 7 + 9 + 10 | |
| 1 + 2 + 3 + 5 + 9 | 2 + 3 + 4 + 7 + 8 | 3 + 4 + 5 + 9 + 10 | 4 + 6 + 7 + 8 + 9 | |
| 1 + 2 + 3 + 5 + 10 | 2 + 3 + 4 + 7 + 9 | 3 + 4 + 6 + 7 + 8 | 4 + 6 + 7 + 8 + 10 | |
| 1 + 2 + 3 + 6 + 7 | 2 + 3 + 4 + 7 + 10 | 3 + 4 + 6 + 7 + 9 | 4 + 6 + 7 + 9 + 10 | |
| 1 + 2 + 3 + 6 + 8 | 2 + 3 + 4 + 8 + 9 | 3 + 4 + 6 + 7 + 10 | 4 + 7 + 8 + 9 + 10 | |
| 1 + 2 + 3 + 6 + 9 | 2 + 3 + 4 + 8 + 10 | 3 + 4 + 6 + 8 + 9 | | |
| 1 + 2 + 3 + 6 + 10 | 2 + 3 + 4 + 9 + 10 | 3 + 4 + 6 + 8 + 10 | | |
| 1 + 2 + 3 + 7 + 8 | 2 + 3 + 5 + 6 + 7 | 3 + 4 + 6 + 9 + 10 | | |
| 1 + 2 + 3 + 7 + 9 | 2 + 3 + 5 + 6 + 8 | 3 + 4 + 7 + 8 + 9 | | |
| 1 + 2 + 3 + 7 + 10 | 2 + 3 + 5 + 6 + 9 | 3 + 4 + 7 + 8 + 10 | | |
| 1 + 2 + 3 + 8 + 9 | 2 + 3 + 5 + 6 + 10 | 3 + 4 + 7 + 9 + 10 | | |
| 1 + 2 + 3 + 8 + 10 | 2 + 3 + 5 + 7 + 8 | 3 + 4 + 8 + 9 + 10 | | |
| 1 + 2 + 3 + 9 + 10 | 2 + 3 + 5 + 7 + 9 | 3 + 5 + 6 + 7 + 8 | | |
| 1 + 2 + 4 + 5 + 6 | 2 + 3 + 5 + 7 + 10 | 3 + 5 + 6 + 7 + 9 | | |
| 1 + 2 + 4 + 5 + 7 | 2 + 3 + 5 + 8 + 9 | 3 + 5 + 6 + 7 + 10 | | |
| 1 + 2 + 4 + 5 + 8 | 2 + 3 + 5 + 8 + 10 | 3 + 5 + 7 + 8 + 9 | | |
| 1 + 2 + 4 + 5 + 9 | 2 + 3 + 5 + 9 + 10 | 3 + 5 + 7 + 8 + 10 | | |
| 1 + 2 + 4 + 5 + 10 | 2 + 3 + 6 + 7 + 8 | 3 + 5 + 7 + 9 + 10 | | |
| 1 + 2 + 4 + 6 + 7 | 2 + 3 + 6 + 7 + 9 | 3 + 5 + 8 + 9 + 10 | | |
| 1 + 2 + 4 + 6 + 8 | 2 + 3 + 6 + 7 + 10 | 3 + 6 + 7 + 8 + 9 | | |
| 1 + 2 + 4 + 6 + 9 | 2 + 3 + 6 + 8 + 9 | 3 + 6 + 7 + 8 + 10 | | |
| 1 + 2 + 4 + 6 + 10 | 2 + 3 + 6 + 8 + 10 | 3 + 6 + 7 + 9 + 10 | | |
| 1 + 2 + 4 + 7 + 8 | 2 + 3 + 6 + 9 + 10 | 3 + 6 + 8 + 9 + 10 | | |
| 1 + 2 + 4 + 7 + 9 | 2 + 3 + 7 + 8 + 9 | 3 + 7 + 8 + 9 + 10 | | |
| 1 + 2 + 4 + 7 + 10 | 2 + 3 + 7 + 8 + 10 | | | |
| 1 + 2 + 4 + 8 + 9 | 2 + 3 + 7 + 9 + 10 | | | |
| 1 + 2 + 4 + 8 + 10 | 2 + 3 + 8 + 9 + 10 | | | |
| 1 + 2 + 4 + 9 + 10 | 2 + 4 + 5 + 6 + 7 | | | |
| 1 + 2 + 5 + 6 + 7 | 2 + 4 + 5 + 6 + 8 | | | |
| 1 + 2 + 5 + 6 + 8 | 2 + 4 + 5 + 6 + 9 | | | |
| 1 + 2 + 5 + 6 + 9 | 2 + 4 + 5 + 6 + 10 | | | |
| 1 + 2 + 5 + 6 + 10 | 2 + 4 + 5 + 7 + 8 | | | |
| 1 + 2 + 5 + 7 + 8 | 2 + 4 + 5 + 7 + 9 | | | |
| 1 + 2 + 5 + 7 + 9 | 2 + 4 + 5 + 7 + 10 | | | |
| 1 + 2 + 5 + 7 + 10 | 2 + 4 + 5 + 8 + 9 | | | |
| 1 + 2 + 5 + 8 + 9 | 2 + 4 + 5 + 8 + 10 | | | |
| 1 + 2 + 5 + 8 + 10 | 2 + 4 + 5 + 9 + 10 | | | |
| 1 + 2 + 5 + 9 + 10 | 2 + 4 + 6 + 7 + 8 | | | |
| 1 + 2 + 6 + 7 + 8 | 2 + 4 + 6 + 7 + 9 | | | |
| 1 + 2 + 6 + 7 + 9 | 2 + 4 + 6 + 7 + 10 | | | |
| 1 + 2 + 6 + 7 + 10 | 2 + 4 + 6 + 8 + 9 | | | |
| 1 + 2 + 6 + 8 + 9 | 2 + 4 + 6 + 8 + 10 | | | |
| 1 + 2 + 6 + 8 + 10 | 2 + 4 + 6 + 9 + 10 | | | |
| 1 + 2 + 6 + 9 + 10 | 2 + 4 + 7 + 8 + 9 | | | |
| 1 + 2 + 7 + 8 + 9 | 2 + 4 + 7 + 8 + 10 | | | |
| 1 + 2 + 7 + 8 + 10 | 2 + 4 + 7 + 9 + 10 | | | |
| 1 + 2 + 7 + 9 + 10 | 2 + 4 + 8 + 9 + 10 | | | |
| 1 + 2 + 8 + 9 + 10 | 2 + 5 + 6 + 7 + 8 | | | |
| 1 + 3 + 4 + 5 + 6 | 2 + 5 + 6 + 7 + 9 | | | |
| 1 + 3 + 4 + 5 + 7 | 2 + 5 + 6 + 7 + 10 | | | |
| 1 + 3 + 4 + 5 + 8 | 2 + 5 + 6 + 8 + 9 | | | |
| 1 + 3 + 4 + 5 + 9 | 2 + 5 + 6 + 8 + 10 | | | |
| 1 + 3 + 4 + 5 + 10 | 2 + 5 + 6 + 9 + 10 | | | |
| 1 + 3 + 4 + 6 + 7 | 2 + 6 + 7 + 8 + 9 | | | |
| 1 + 3 + 4 + 6 + 8 | 2 + 6 + 7 + 8 + 10 | | | |
| 1 + 3 + 4 + 6 + 9 | 2 + 6 + 8 + 9 + 10 | | | |
| 1 + 3 + 4 + 6 + 10 | 2 + 7 + 8 + 9 + 10 | | | |
| 1 + 3 + 4 + 7 + 8 | | | | |
| 1 + 3 + 4 + 7 + 9 | | | | |
| 1 + 3 + 4 + 7 + 10 | | | | |
| 1 + 3 + 4 + 8 + 9 | | | | |
| 1 + 3 + 4 + 8 + 10 | | | | |
| 1 + 3 + 4 + 9 + 10 | | | | |
| 1 + 3 + 5 + 6 + 7 | | | | |
| 1 + 3 + 5 + 6 + 8 | | | | |
| 1 + 3 + 5 + 6 + 9 | | | | |
| 1 + 3 + 5 + 6 + 10 | | | | |
| 1 + 3 + 5 + 7 + 8 | | | | |
| 1 + 3 + 5 + 7 + 9 | | | | |
| 1 + 3 + 5 + 7 + 10 | | | | |
| 1 + 3 + 5 + 8 + 9 | | | | |
| 1 + 3 + 5 + 8 + 10 | | | | |
| 1 + 3 + 5 + 9 + 10 | | | | |
| 1 + 3 + 6 + 7 + 8 | | | | |
| 1 + 3 + 6 + 7 + 9 | | | | |
| 1 + 3 + 6 + 7 + 10 | | | | |
| 1 + 3 + 6 + 8 + 9 | | | | |
| 1 + 3 + 6 + 8 + 10 | | | | |
| 1 + 3 + 6 + 9 + 10 | | | | |
| 1 + 4 + 5 + 6 + 7 | | | | |
| 1 + 4 + 5 + 6 + 8 | | | | |
| 1 + 4 + 5 + 6 + 9 | | | | |
| 1 + 4 + 5 + 6 + 10 | | | | |
| 1 + 4 + 5 + 7 + 8 | | | | |
| 1 + 4 + 5 + 7 + 9 | | | | |
| 1 + 4 + 5 + 7 + 10 | | | | |
| 1 + 4 + 5 + 8 + 9 | | | | |
| 1 + 4 + 5 + 8 + 10 | | | | |
| 1 + 4 + 5 + 9 + 10 | | | | |
| 1 + 4 + 6 + 7 + 8 | | | | |
| 1 + 4 + 6 + 7 + 9 | | | | |
| 1 + 4 + 6 + 7 + 10 | | | | |
| 1 + 4 + 6 + 8 + 9 | | | | |
| 1 + 4 + 6 + 8 + 10 | | | | |
| 1 + 4 + 6 + 9 + 10 | | | | |
| 1 + 5 + 6 + 7 + 8 | | | | |
| 1 + 5 + 6 + 7 + 9 | | | | |
| 1 + 5 + 6 + 7 + 10 | | | | |
| 1 + 5 + 6 + 8 + 9 | | | | |
| 1 + 5 + 6 + 8 + 10 | | | | |
| 1 + 5 + 6 + 9 + 10 | | | | |
| 1 + 5 + 7 + 8 + 9 | | | | |
| 1 + 5 + 7 + 8 + 10 | | | | |
| 1 + 5 + 7 + 9 + 10 | | | | |
| 1 + 5 + 8 + 9 + 10 | | | | |
| 1 + 6 + 7 + 8 + 9 | | | | |
| 1 + 6 + 7 + 8 + 10 | | | | |
| 1 + 6 + 7 + 9 + 10 | | | | |
| 1 + 6 + 8 + 9 + 10 | | | | |
| 1 + 7 + 8 + 9 + 10 | | | | |

Table Five key -
1: Anti-DIG antibody#/DIG*
2: Anti-Texas Red antibody#/Texas Red*
3: Anti-Cascade Blue antibody#/Cascade Blue*
4: Anti-TAMRA antibody#/TAMRA*
5: Anti-Dinitrophenol antibody#/Dinitrophenol*
6: Anti-Cy5 antibody#/Cy5*
7: Anti-Dansyl antibody#/Dansyl*
8: Anti-Streptavidin antibody#/Streptavidin*
9: Anti-Biotin antibody#/Biotin*
10: Strepatividin#/Biotin*
#includes antigen-binding fragments and derivatives thereof
*includes fragments and derivatives thereof

The binding molecule of any one, any two, any three, any four, or all five pairs may be immobilised in a detection zone of the LFD. Alternatively, the binding molecule of any one, any two, any three, any four, or all five pairs may be directly or indirectly bound to a signalling molecule.

In some embodiments of the present invention, the LFD may comprise a combination of five binding molecule/ligand pairs shown in Table 6 below.

TABLE SIX
combinations of six binding molecule/ligand pairs

| | | | |
|---|---|---|---|
| 1 + 2 + 3 + 4 + 5 + 6 | 2 + 3 + 4 + 5 + 6 + 7 | 3 + 4 + 5 + 6 + 7 + 8 | 4 + 5 + 6 + 7 + 8 + 9 |
| 1 + 2 + 3 + 4 + 5 + 7 | 2 + 3 + 4 + 5 + 6 + 8 | 3 + 4 + 5 + 6 + 7 + 9 | 4 + 5 + 6 + 7 + 8 + 10 |
| 1 + 2 + 3 + 4 + 5 + 8 | 2 + 3 + 4 + 5 + 6 + 9 | 3 + 4 + 5 + 6 + 7 + 10 | 4 + 5 + 6 + 7 + 9 + 10 |
| 1 + 2 + 3 + 4 + 5 + 9 | 2 + 3 + 4 + 5 + 6 + 10 | 3 + 4 + 5 + 6 + 8 + 9 | 4 + 5 + 6 + 8 + 9 + 10 |
| 1 + 2 + 3 + 4 + 5 + 10 | 2 + 3 + 4 + 5 + 7 + 8 | 3 + 4 + 5 + 6 + 8 + 10 | 4 + 5 + 7 + 8 + 9 + 10 |
| 1 + 2 + 3 + 4 + 6 + 7 | 2 + 3 + 4 + 5 + 7 + 9 | 3 + 4 + 5 + 6 + 9 + 10 | 4 + 6 + 7 + 8 + 9 + 10 |
| 1 + 2 + 3 + 4 + 6 + 8 | 2 + 3 + 4 + 5 + 7 + 10 | 3 + 4 + 5 + 7 + 8 + 9 | |
| 1 + 2 + 3 + 4 + 6 + 9 | 2 + 3 + 4 + 5 + 8 + 9 | 3 + 4 + 5 + 7 + 8 + 10 | 5 + 6 + 7 + 8 + 9 + 10 |
| 1 + 2 + 3 + 4 + 6 + 10 | 2 + 3 + 4 + 5 + 8 + 10 | 3 + 4 + 5 + 7 + 9 + 10 | |
| 1 + 2 + 3 + 4 + 7 + 8 | 2 + 3 + 4 + 5 + 9 + 10 | 3 + 4 + 5 + 8 + 9 + 10 | |
| 1 + 2 + 3 + 4 + 7 + 9 | 2 + 3 + 4 + 6 + 7 + 8 | 3 + 4 + 6 + 7 + 8 + 9 | |
| 1 + 2 + 3 + 4 + 7 + 10 | 2 + 3 + 4 + 6 + 7 + 9 | 3 + 4 + 6 + 7 + 8 + 10 | |
| 1 + 2 + 3 + 4 + 8 + 9 | 2 + 3 + 4 + 6 + 7 + 10 | 3 + 4 + 6 + 7 + 9 + 10 | |
| 1 + 2 + 3 + 4 + 8 + 10 | 2 + 3 + 4 + 6 + 8 + 9 | 3 + 4 + 6 + 8 + 9 + 10 | |
| 1 + 2 + 3 + 4 + 9 + 10 | 2 + 3 + 4 + 6 + 8 + 10 | 3 + 4 + 7 + 8 + 9 + 10 | |
| 1 + 2 + 3 + 5 + 6 + 7 | 2 + 3 + 4 + 6 + 9 + 10 | 3 + 5 + 6 + 7 + 8 + 9 | |
| 1 + 2 + 3 + 5 + 6 + 8 | 2 + 3 + 4 + 7 + 8 + 9 | 3 + 5 + 6 + 7 + 8 + 10 | |
| 1 + 2 + 3 + 5 + 6 + 9 | 2 + 3 + 4 + 7 + 8 + 10 | 3 + 5 + 6 + 7 + 9 + 10 | |
| 1 + 2 + 3 + 5 + 6 + 10 | 2 + 3 + 4 + 7 + 9 + 10 | 3 + 5 + 7 + 8 + 9 + 10 | |
| 1 + 2 + 3 + 5 + 7 + 8 | 2 + 3 + 4 + 8 + 9 + 10 | 3 + 6 + 7 + 8 + 9 + 10 | |
| 1 + 2 + 3 + 5 + 7 + 9 | 2 + 3 + 5 + 6 + 7 + 8 | | |
| 1 + 2 + 3 + 5 + 7 + 10 | 2 + 3 + 5 + 6 + 7 + 9 | | |
| 1 + 2 + 3 + 5 + 8 + 9 | 2 + 3 + 5 + 6 + 7 + 10 | | |
| 1 + 2 + 3 + 5 + 8 + 10 | 2 + 3 + 5 + 6 + 8 + 9 | | |
| 1 + 2 + 3 + 5 + 9 + 10 | 2 + 3 + 5 + 6 + 8 + 10 | | |
| 1 + 2 + 3 + 6 + 7 + 8 | 2 + 3 + 5 + 6 + 9 + 10 | | |
| 1 + 2 + 3 + 6 + 7 + 9 | 2 + 3 + 5 + 7 + 8 + 9 | | |
| 1 + 2 + 3 + 6 + 7 + 10 | 2 + 3 + 5 + 7 + 8 + 10 | | |
| 1 + 2 + 3 + 6 + 8 + 9 | 2 + 3 + 5 + 7 + 9 + 10 | | |
| 1 + 2 + 3 + 6 + 8 + 10 | 2 + 3 + 5 + 8 + 9 + 10 | | |
| 1 + 2 + 3 + 6 + 9 + 10 | 2 + 3 + 6 + 7 + 8 + 9 | | |
| 1 + 2 + 3 + 7 + 8 + 9 | 2 + 3 + 6 + 7 + 8 + 10 | | |
| 1 + 2 + 3 + 7 + 8 + 10 | 2 + 3 + 6 + 7 + 9 + 10 | | |
| 1 + 2 + 3 + 7 + 9 + 10 | 2 + 3 + 6 + 8 + 9 + 10 | | |
| 1 + 2 + 3 + 8 + 9 + 10 | 2 + 3 + 7 + 8 + 9 + 10 | | |
| 1 + 2 + 4 + 5 + 6 + 7 | 2 + 4 + 5 + 6 + 7 + 8 | | |
| 1 + 2 + 4 + 5 + 6 + 8 | 2 + 4 + 5 + 6 + 7 + 9 | | |
| 1 + 2 + 4 + 5 + 6 + 9 | 2 + 4 + 5 + 6 + 7 + 10 | | |
| 1 + 2 + 4 + 5 + 6 + 10 | 2 + 4 + 5 + 6 + 8 + 9 | | |
| 1 + 2 + 4 + 6 + 7 + 8 | 2 + 4 + 5 + 6 + 8 + 10 | | |
| 1 + 2 + 4 + 6 + 7 + 9 | 2 + 4 + 5 + 6 + 9 + 10 | | |
| 1 + 2 + 4 + 6 + 7 + 10 | 2 + 4 + 5 + 7 + 8 + 9 | | |
| 1 + 2 + 4 + 6 + 8 + 9 | 2 + 4 + 5 + 7 + 8 + 10 | | |
| 1 + 2 + 4 + 6 + 8 + 10 | 2 + 4 + 5 + 7 + 9 + 10 | | |
| 1 + 2 + 4 + 6 + 9 + 10 | 2 + 4 + 5 + 8 + 9 + 10 | | |
| 1 + 2 + 4 + 7 + 8 + 9 | 2 + 4 + 6 + 7 + 8 + 9 | | |
| 1 + 2 + 4 + 7 + 8 + 10 | 2 + 4 + 6 + 7 + 8 + 10 | | |
| 1 + 2 + 4 + 7 + 9 + 10 | 2 + 4 + 6 + 7 + 9 + 10 | | |
| 1 + 2 + 4 + 8 + 9 + 10 | 2 + 4 + 6 + 8 + 9 + 10 | | |
| 1 + 2 + 5 + 6 + 7 + 8 | 2 + 4 + 7 + 8 + 9 + 10 | | |
| 1 + 2 + 5 + 6 + 7 + 9 | 2 + 5 + 6 + 7 + 8 + 9 | | |
| 1 + 2 + 5 + 6 + 7 + 10 | 2 + 5 + 6 + 7 + 8 + 10 | | |
| 1 + 2 + 5 + 7 + 8 + 9 | 2 + 5 + 6 + 7 + 9 + 10 | | |
| 1 + 2 + 5 + 7 + 8 + 10 | 2 + 5 + 6 + 8 + 9 + 10 | | |
| 1 + 2 + 5 + 7 + 9 + 10 | 2 + 5 + 7 + 8 + 9 + 10 | | |
| 1 + 2 + 5 + 8 + 9 + 10 | 2 + 6 + 7 + 8 + 9 + 10 | | |
| 1 + 2 + 6 + 7 + 8 + 9 | | | |
| 1 + 2 + 6 + 7 + 8 + 10 | | | |
| 1 + 2 + 6 + 7 + 9 + 10 | | | |
| 1 + 2 + 7 + 8 + 9 + 10 | | | |
| 1 + 3 + 4 + 5 + 6 + 7 | | | |
| 1 + 3 + 4 + 5 + 6 + 8 | | | |
| 1 + 3 + 4 + 5 + 6 + 9 | | | |
| 1 + 3 + 4 + 5 + 6 + 10 | | | |
| 1 + 3 + 4 + 6 + 7 + 8 | | | |
| 1 + 3 + 4 + 6 + 7 + 9 | | | |
| 1 + 3 + 4 + 6 + 7 + 10 | | | |
| 1 + 3 + 4 + 7 + 8 + 9 | | | |
| 1 + 3 + 4 + 7 + 8 + 10 | | | |
| 1 + 3 + 4 + 8 + 9 + 10 | | | |
| 1 + 3 + 5 + 6 + 7 + 8 | | | |
| 1 + 3 + 5 + 6 + 7 + 9 | | | |
| 1 + 3 + 5 + 6 + 7 + 10 | | | |
| 1 + 3 + 5 + 7 + 8 + 9 | | | |
| 1 + 3 + 5 + 7 + 8 + 10 | | | |
| 1 + 3 + 5 + 7 + 9 + 10 | | | |
| 1 + 3 + 6 + 7 + 8 + 9 | | | |
| 1 + 3 + 6 + 7 + 8 + 10 | | | |
| 1 + 3 + 6 + 7 + 9 + 10 | | | |
| 1 + 3 + 7 + 8 + 9 + 10 | | | |
| 1 + 4 + 5 + 6 + 7 + 8 | | | |
| 1 + 4 + 5 + 6 + 7 + 9 | | | |
| 1 + 4 + 5 + 6 + 7 + 10 | | | |
| 1 + 4 + 5 + 7 + 8 + 9 | | | |
| 1 + 4 + 5 + 7 + 8 + 10 | | | |
| 1 + 4 + 5 + 8 + 9 + 10 | | | |
| 1 + 4 + 6 + 7 + 8 + 9 | | | |
| 1 + 4 + 6 + 7 + 8 + 10 | | | |
| 1 + 4 + 6 + 8 + 9 + 10 | | | |
| 1 + 5 + 6 + 7 + 8 + 9 | | | |
| 1 + 5 + 6 + 7 + 8 + 10 | | | |
| 1 + 5 + 7 + 8 + 9 + 10 | | | |
| 1 + 6 + 7 + 8 + 9 + 10 | | | |

Table Six key -
1: Anti-DIG antibody#/DIG*
2: Anti-Texas Red antibody#/Texas Red*
3: Anti-Cascade Blue antibody#/Cascade Blue*
4: Anti-TAMRA antibody#/TAMRA*
5: Anti-Dinitrophenol antibody#/Dinitrophenol*
6: Anti-Cy5 antibody#/Cy5*
7: Anti-Dansyl antibody#/Dansyl*
8: Anti-Streptavidin antibody#/Streptavidin*
9: Anti-Biotin antibody#/Biotin*
10: Strepatividin#/Biotin*
#includes antigen-binding fragments and derivatives thereof
*includes fragments and derivatives thereof

The binding molecule of any one, any two, any three, any four, any five, or all six pairs may be immobilised in a detection zone of the LFD. Alternatively, the binding molecule of any one, any two, any three, any four, any five, or all six pairs may be directly or indirectly bound to a signalling molecule.

In some embodiments of the present invention, the LFD may comprise a combination of seven binding molecule/ligand pairs shown in Table 7 below.

TABLE SEVEN
combinations of seven binding molecule/ligand pairs

| | | |
|---|---|---|
| 1 + 2 + 3 + 4 + 5 + 6 + 7 | 2 + 3 + 4 + 5 + 6 + 7 + 8 | 3 + 4 + 5 + 6 + 7 + 8 + 9 |
| 1 + 2 + 3 + 4 + 5 + 6 + 8 | 2 + 3 + 4 + 5 + 6 + 7 + 9 | 3 + 4 + 5 + 6 + 7 + 8 + 10 |
| 1 + 2 + 3 + 4 + 5 + 6 + 9 | 2 + 3 + 4 + 5 + 6 + 7 + 10 | 3 + 4 + 5 + 6 + 7 + 9 + 10 |
| 1 + 2 + 3 + 4 + 5 + 6 + 10 | 2 + 3 + 4 + 5 + 6 + 8 + 9 | 3 + 4 + 5 + 6 + 8 + 9 + 10 |
| 1 + 2 + 3 + 4 + 5 + 7 + 8 | 2 + 3 + 4 + 5 + 6 + 8 + 10 | 3 + 4 + 5 + 7 + 8 + 9 + 10 |
| 1 + 2 + 3 + 4 + 5 + 7 + 9 | 2 + 3 + 4 + 5 + 6 + 9 + 10 | 3 + 4 + 6 + 7 + 8 + 9 + 10 |
| 1 + 2 + 3 + 4 + 5 + 7 + 10 | 2 + 3 + 4 + 5 + 7 + 8 + 9 | 3 + 5 + 6 + 7 + 8 + 9 + 10 |
| 1 + 2 + 3 + 4 + 5 + 8 + 9 | 2 + 3 + 4 + 5 + 7 + 8 + 10 | |
| 1 + 2 + 3 + 4 + 5 + 8 + 10 | 2 + 3 + 4 + 5 + 7 + 9 + 10 | 4 + 5 + 6 + 7 + 8 + 9 + 10 |
| 1 + 2 + 3 + 4 + 5 + 9 + 10 | 2 + 3 + 4 + 5 + 8 + 9 + 10 | |
| 1 + 2 + 3 + 4 + 6 + 7 + 8 | 2 + 3 + 4 + 6 + 7 + 8 + 9 | |
| 1 + 2 + 3 + 4 + 6 + 7 + 9 | 2 + 3 + 4 + 6 + 7 + 8 + 10 | |
| 1 + 2 + 3 + 4 + 6 + 7 + 10 | 2 + 3 + 4 + 6 + 7 + 9 + 10 | |
| 1 + 2 + 3 + 4 + 6 + 8 + 9 | 2 + 3 + 4 + 6 + 8 + 9 + 10 | |
| 1 + 2 + 3 + 4 + 6 + 8 + 10 | 2 + 3 + 4 + 7 + 8 + 9 + 10 | |
| 1 + 2 + 3 + 4 + 6 + 9 + 10 | 2 + 3 + 5 + 6 + 7 + 8 + 9 | |
| 1 + 2 + 3 + 4 + 7 + 8 + 9 | 2 + 3 + 5 + 6 + 7 + 8 + 10 | |
| 1 + 2 + 3 + 4 + 7 + 8 + 10 | 2 + 3 + 5 + 6 + 7 + 9 + 10 | |
| 1 + 2 + 3 + 4 + 7 + 9 + 10 | 2 + 3 + 5 + 6 + 8 + 9 + 10 | |
| 1 + 2 + 3 + 4 + 8 + 9 + 10 | 2 + 3 + 5 + 7 + 8 + 9 + 10 | |
| 1 + 2 + 3 + 5 + 6 + 7 + 8 | 2 + 3 + 6 + 7 + 8 + 9 + 10 | |
| 1 + 2 + 3 + 5 + 6 + 7 + 9 | | |
| 1 + 2 + 3 + 5 + 6 + 7 + 10 | | |
| 1 + 2 + 3 + 5 + 6 + 8 + 9 | | |
| 1 + 2 + 3 + 5 + 6 + 8 + 10 | | |
| 1 + 2 + 3 + 5 + 6 + 9 + 10 | | |
| 1 + 2 + 3 + 5 + 7 + 8 + 9 | | |
| 1 + 2 + 3 + 5 + 7 + 8 + 10 | | |
| 1 + 2 + 3 + 5 + 7 + 9 + 10 | | |
| 1 + 2 + 3 + 5 + 8 + 9 + 10 | | |
| 1 + 2 + 3 + 6 + 7 + 8 + 9 | | |
| 1 + 2 + 3 + 6 + 7 + 8 + 10 | | |
| 1 + 2 + 3 + 6 + 7 + 9 + 10 | | |
| 1 + 2 + 3 + 6 + 8 + 9 + 10 | | |
| 1 + 2 + 3 + 7 + 8 + 9 + 10 | | |
| 1 + 2 + 4 + 6 + 7 + 8 + 9 | | |
| 1 + 2 + 4 + 6 + 7 + 8 + 10 | | |
| 1 + 2 + 4 + 6 + 7 + 9 + 10 | | |
| 1 + 2 + 4 + 6 + 8 + 9 + 10 | | |
| 1 + 2 + 5 + 6 + 7 + 8 + 9 | | |
| 1 + 2 + 5 + 6 + 7 + 8 + 10 | | |
| 1 + 2 + 5 + 6 + 7 + 9 + 10 | | |
| 1 + 2 + 5 + 6 + 8 + 9 + 10 | | |
| 1 + 2 + 5 + 7 + 8 + 9 + 10 | | |
| 1 + 2 + 6 + 7 + 8 + 9 + 10 | | |
| 1 + 3 + 4 + 5 + 6 + 7 + 8 | | |
| 1 + 3 + 4 + 5 + 6 + 7 + 9 | | |
| 1 + 3 + 4 + 5 + 6 + 7 + 10 | | |
| 1 + 3 + 4 + 5 + 6 + 8 + 9 | | |
| 1 + 3 + 4 + 5 + 6 + 8 + 10 | | |
| 1 + 3 + 4 + 5 + 6 + 9 + 10 | | |
| 1 + 3 + 4 + 5 + 7 + 8 + 9 | | |
| 1 + 3 + 4 + 5 + 7 + 8 + 10 | | |
| 1 + 3 + 4 + 5 + 7 + 9 + 10 | | |
| 1 + 3 + 4 + 5 + 8 + 9 + 10 | | |
| 1 + 3 + 4 + 6 + 7 + 8 + 9 | | |
| 1 + 3 + 4 + 6 + 7 + 8 + 10 | | |
| 1 + 3 + 4 + 6 + 7 + 9 + 10 | | |
| 1 + 3 + 4 + 7 + 8 + 9 + 10 | | |
| 1 + 3 + 5 + 6 + 7 + 8 + 9 | | |
| 1 + 3 + 5 + 6 + 7 + 8 + 10 | | |
| 1 + 3 + 5 + 6 + 7 + 9 + 10 | | |
| 1 + 3 + 5 + 6 + 8 + 9 + 10 | | |
| 1 + 3 + 5 + 7 + 8 + 9 + 10 | | |
| 1 + 3 + 6 + 7 + 8 + 9 + 10 | | |

Table Seven key -
1: Anti-DIG antibody#/DIG*
2: Anti-Texas Red antibody#/Texas Red*
3: Anti-Cascade Blue antibody#/Cascade Blue*
4: Anti-TAMRA antibody#/TAMRA*
5: Anti-Dinitrophenol antibody#/Dinitrophenol*
6: Anti-Cy5 antibody#/Cy5*
7: Anti-Dansyl antibody#/Dansyl*
8: Anti-Streptavidin antibody#/Streptavidin*
9: Anti-Biotin antibody#/Biotin*
10: Strepatividin#/Biotin*
4 includes antigen-binding fragments and derivatives thereof
* includes fragments and derivatives thereof
#includes antigen-binding fragments and derivatives thereof
*includes fragments and derivatives thereof

The binding molecule of any one, any two, any three, any four, any five, any six, or all seven pairs may be immobilised in a detection zone of the LFD. Alternatively, the binding molecule of any one, any two, any three, any four, any five, any six, or all seven pairs may be directly or indirectly bound to a signalling molecule.

Signalling Molecules

Signalling molecules according to the present invention, also known in the art as tracers, may include any molecules capable of providing a detectable signal which can be indicative of the presence of a target analyte.

The signalling molecule may be a component of a signalling complex.

For example, the signalling complex may comprise a target analyte bound to a ligand which is in turn bound to a binding molecule having binding specificity for the ligand. The signalling molecule may be bound to the target analyte itself and/or the binding molecule.

Alternatively the signalling complex may, for example, comprise a target analyte bound to a first ligand which is in turn bound to a first binding molecule having binding specificity for the ligand. The analyte may also be bound to a second ligand which is in turn bound to a second binding molecule which has binding specificity for the second ligand and is of a different type to the first binding molecule.

Alternatively, the signalling complex may, for example, comprise a target analyte labelled with a ligand via an antibody or aptamer that has binding specificity for the analyte, and to which the ligand is bound. One portion of the aptamer or antibody (e.g. via the Fc region) can be bound to the ligand, while another portion can be bound to the analyte. This arrangement may be duplicated such that the analyte is bound to two antibodies, two aptamers, or an antibody and an aptamer, which link the analyte to two different types of ligands. One ligand may be bound by the detection molecule while the second may be bound by a capture molecule (e.g. an antibody or aptamer) capable of providing a detectable signal (e.g. via a conjugated nanoparticle, enzyme, fluorescent label etc.)

Methods for preparing and utilising signalling molecules are well known in the art, and exemplified in the Examples of the present application.

Non limiting examples of suitable signalling molecules include enzymes, enzyme substrates, beads, particles, fluorescent dyes, nanomaterials (e.g. latex beads or colloidal gold particles, carbon particles, magnetic particles, paramagnetic particles, quantum dots, up-converting phosphorus, nano microspheres, nano-tubes, chelate-loaded silica, europium), liposomes, fluorescent immunoliposomes, and the like.

Without any particular limitation being imparted, the signalling molecule may provide a colourimetric signal. By way of non-limiting example, either of the following methods may be applied to quantify results: (1) using a (hand-held) lateral flow strip reader; (2) photo taking equipment (e.g. camera, or mobile phone) coupled with image software analysis.

Again without any particular limitation being imparted, the signalling molecule may provide a signal selected from: fluorescence, chemifluorescence, chemiluminescence, near-infrared fluorescence, a magnetic signal, an electrical signal. Specific equipment to measure these signals are well known to those of ordinary skill in the art.

Analytes

Analytes for detection according to the present invention include, but are not limited to, nucleic acids, proteins, peptides, lipids, small molecules, glycoproteins, pathogens, lipids, lipoproteins, cells, metabolites, viruses, metal ions, archaea, fungi, bacteria, prions, toxins, antibodies, contaminants, entire organisms, poisons, polymers, metal salts, and derivatives of any one of the aforementioned.

Any type of nucleic acids may be detected including, again without limitation, DNA, RNA and cDNA. The analytes may be natural or synthetic (e.g. amplified nucleic acids).

In some embodiments, the nucleic acids are subjected to amplification prior to application to the LFD, for example by thermal amplification and/or by isothermal amplification. Non-limiting examples of suitable amplification techniques include polymerase chain reaction (PCR), strand displacement amplification (SDA), loop-mediated isothermal amplification (LAMP), rolling circle amplification (RCA), transcription-mediated amplification (TMA), self-sustained sequence replication (3 SR), nucleic acid sequence based amplification (NASBA), reverse transcription polymerase chain reaction (RT-PCR), recombinase polymerase amplification (RPA), loop mediated isothermal amplification (LAMP), helicase-dependent amplification (HDA) and the like.

In the case of nucleic acids, primers and/or probes used in the amplification of the nucleic acids may be labelled with a ligand, such that amplification culminates in the production of amplicons incorporating the ligands by virtue of the primers/probes used. The present inventors have identified that a range of new detection molecules can function as ligands. In some embodiments, primers conjugated to these ligands may be incorporated during, for example, an RPA reaction.

Ligands may also be indirectly bound to target analytes (e.g. proteins, peptides, lipids, small molecules) via aptamers or antibodies which are themselves directly bound to ligands. The aptamers may comprise nucleic acids and/or amino acids (e.g. peptide aptamers). The aptamer or antibody may have binding specificity for the target analyte, and consequently provide a link between the target analyte and the ligand both of which are bound by the antibody or aptamer.

Immobilisation of Molecules

Molecules may be immobilised in one or more zones of the LFD.

For example, antibodies can be adsorbed onto tracers (e.g. nanomaterials) or membranes via electrostatic, hydrophobic, van der Waals and/or hydrogen forces. Optimally, the antigenic binding sites can be fully exposed towards the analyte. Non-limiting examples of methods by which this may be achieved include bioaffinity and by chemically forming covalent bonds. Bioaffinity requires a strong ligand pair (e.g. biotin and avidin), and the approach may include biotinylating the antibody in a site-specific manner (e.g. to the Fc region), before immobilisation via pre-immobilised avidin. The chemical approach can be performed by applying specific conjugation chemistries (e.g. carbodiimide and maleimide) between certain functional groups of the antibody (e.g. amine, carboxylate or sulfhydryl groups) and a functionalised surface of the nanomaterial or membrane. The carboxylate group in the Fc region and the sulfhydryl groups in the hinge and interchain regions of an antibody are suitable conjugation sites. Linkers (e.g. polyethylene glycol, PEG) may be applied in between the biomolecule and the surface of the tracer or membrane to minimise steric hindrance. The use of protein A or G immobilisation which bind specifically to the Fc portion of immunoglobulin from many mammals may be used to assist in orienting the antibodies to maximise the binding activity of an antibody.

Immobilisation of nucleic acids to the tracer or membrane surfaces may be performed using surface chemistry. Nucleic acids can be readily immobilised by forming bonds via amine, sulfhydryl or cyanide groups (tethered to one end of nucleic acid). Accordingly, the surface chemistry described above for antibody immobilisation can be similarly applied for nucleic acid immobilisation. For both antibody and nucleic acids immobilised to the tracer or membrane, blocking reagents such as Tween 20, BSA or casein may be used to block non-specific binding of the analyte, which may be performed after immobilisation.

Additional LFD Components

LFD according to the present invention may include any number of standard components, non-limiting examples of which include membranes, pads, plastic housing, and the like.

A membrane for use in an LFD according to the present invention may serve as a stable binding surface to capture binding molecules (e.g. antibodies) onto the test and control lines, and/or to control the diffusive and capillary flow of the mobile phase (running buffer). The membrane may comprise any suitable pore size, capillary flow rate, porosity and thickness. Non-limiting examples of suitable materials from which the membranes may be made include nitrocellulose, nylon, polyethersulfone, polyethylene, polyvinylidine difluoride (PVDF) or fused silica.

The LFD may comprise one or a series of interconnected pads that are mounted on a backing card. For example, the LFD may comprise a sample pad. The sample pad may ensure even distribution of sample solution to upstream components. By way of non-limiting example, the sample pad can be made of cellulose or glass fiber, and may be at the site of sample loading. Reagents such as detergents (e.g. Tween or Triton) and blocking solution (e.g. bovine serum albumin, BSA or polyvinyl alcohol) can be impregnated within the sample pad to enhance the flow rate. The sample pad may also serve to pretreat of the sample to remove coarse materials (e.g. the whole cell).

Additionally or alternatively, the LFD may comprise a conjugate pad. The conjugate pad may be adhered next to the sample pad, and may be the location in the LFD where labelled detection molecules and/or tracer conjugates are deposited. The primary main function of the conjugate pad may be to control the release of reactants (e.g. analyte/antibody/tracer complexes) solution onto the membrane and/or to hold the reactants stable over their entire shelf-life. The conjugate pad can be made, for example, of glass fiber, polyester or synthetic non-absorbent material (e.g. rayon).

Additionally or alternatively, the LFD may comprise incubation and detection pads that are applied to the back of the membrane, which may assist in stabilising the membrane. The incubation and detection pads may be may be mounted on a backing card.

Additionally or alternatively, the LFD may comprise an absorbent pad. The absorbent pad may enhance the capillary driving force and/or absorb all the unreacted substances. By way of non-limiting example, the LFD may be made of cotton and the absorbency rate may vary with different thicknesses.

The LFD may comprise running buffer, which may interact with all the molecules (analyte, detection molecules and capture/tracer molecules) involved in the lateral flow immunoassay. Non-limiting examples of suitable buffers include phosphate-buffered saline (PBS), tris-buffered saline (TBS), and borate buffer. The buffer may comprise blocking reagent/s. A suitable pH range for the buffer may be between 7.5 and 8.8. LFDs relying on oligonucleotide hybridisations may apply higher stringency buffers such as, for example, saline sodium citrate (SSC).

An LFD device according to the present invention may be sealed in a suitable housing (e.g. plastic housing), with the exception of the sample inlet window and/or the result reading window.

An LFD device according to the present invention will typically comprise different zones. By way of non-limiting example only, the LFD device may comprise any one or more of a sample application zone/inlet, a zone comprising capture molecules and/or tracer molecules that may or may not be immobilised, a test/reaction zone comprising immobilised capture molecules or in some cases immobilised labelled analytes, and a control zone comprising immobilised molecules capable of indicating whether the sample has migrated through the device as intended, regardless of whether the analyte is present in the sample.

Exemplary LFD Formats and Assays

LFDs and methods for their use according to the present invention may adopt a variety of different formats. The following are exemplary formats and not limiting on the scope of the invention.

### Non Competitive Assays

In some embodiments, non-competitive immunoassays are utilised otherwise known as a sandwich assay or sandwich immunoassay. In this setup, the analyte may be embedded between the capture and detection molecules (e.g. antibodies or aptamers), and the detection signal is proportional to the increasing concentration of the analyte.

For example, different binding molecule populations (e.g. detection antibodies or aptamers) may be immobilised in a detection zone of the LFD in a spatially separated manner. Some or all of the binding molecule populations may have binding specificity for a different ligand.

Target analytes may be labelled with one or more ligands prior to exposure to the detection zone.

If the target analyte is a nucleic acid, a ligand may be incorporated at, for example, the 5′ and/or 3′ ends. The ligand at each end of the nucleic acid may be a different type of ligand. Without limitation to a particular method, the ligand/s may be incorporated by utilising primer/s and/or probe/s labelled with the ligand/s such that amplification of the target nucleic acid (e.g. by PCR and/or by isothermal amplification) generates amplicons with ligand attached to the 5′ and/or 3′ ends. One ligand incorporated into the nucleic acid analyte may be bound by members of a detection molecule population, whereas a second (different) ligand may be bound by a capture molecule (e.g. an antibody or aptamer) capable of providing a detectable signal (e.g. via a conjugated tracer such as conjugated nanoparticle, enzyme, fluorescent label etc.).

Alternatively, if the target analyte is a protein, peptide, small molecule, lipid or other non-nucleic acid molecule, the target analyte may be indirectly labelled with a ligand via an antibody or aptamer that has binding specificity for the analyte and to which the ligand is bound. In this case, one portion of the aptamer or antibody (e.g. via the Fc region) can be bound to the ligand, while another portion can be bound to the analyte. This arrangement may be duplicated such that the analyte is bound to two antibodies, two aptamers, or an antibody and an aptamer, that link the analyte to two different types of ligands. One ligand may be bound by the detection molecule while the second may be bound by a capture molecule (e.g. an antibody or aptamer) capable of providing a detectable signal (e.g. via a conjugated tracer such as conjugated nanoparticle, enzyme, fluorescent label etc.).

In the case of a competitive (sandwich) assay, capture molecules may be housed in a conjugate zone of the LFD and, as sample containing the labelled analytes passes through the capture molecules, may move into the detection zone with the labelled analytes allowing signal complexes to form in the detection zone.

A non-competitive format according to the present invention may be capable of detecting one, two, three, four, five, six, or seven different analyte types during operation. For example, when seven target analytes are to be detected, each different type of analyte may be labelled with first and second ligands as set out above. The first ligand can be bound by a detection molecule whereas the second ligand can be bound by a capture molecule. The first ligand associated with any one type of analyte is different from the first ligand associated with any other type/s of analytes. The second ligand associated with all different types of analytes can be the same, or may be different.

The LFD may further comprise a positive control zone. The positive control zone may comprise at least one detection molecule population (e.g. antibodies and/or aptamers) that does not have binding specificity for any of the ligands associated with the target analytes, but instead has binding specificity for the capture molecule. In this way, the positive control can provide indication of whether the capture molecule is capable of providing a detectable signal upon retention in the LFD.

### Competitive Assays

In some embodiments, competitive immunoassays are utilised in which an unknown concentration of analyte competes with a known concentration of the same analyte for binding to detection molecules. The detection signal is inversely proportional to the analyte (unknown concentration).

In one form of this assay, a known amount of analyte may be immobilised in the detection zone. The immobilised analyte may be bound to a ligand, either directly (e.g. a nucleic acid analyte) or via a binding molecule (e.g. an antibody or aptamer) that is bound to the ligand and had binding specificity for the analyte. Various populations of different analyte types may be immobilised in the device in a spatially separated manner. A given analyte type to be quantified in the device is labelled with the same ligand used to label its corresponding analyte population immobilised in the device, and is labelled in the same manner as analytes of that immobilised population. Upon application of the analytes to be quantified and capture molecule population/s with binding specificity for the ligand, the intensity of signal generated from capture molecules bound to the immobilised analytes can be compared to the signal generated from capture molecules bound to the analytes applied to the device (i.e. the analytes of unknown concentration), and the concentration of unknown analytes calculated. The device may comprise a positive control. The positive control may be indicative of binding of the capture molecule to the immobilised analyte (of known amount). A competitive format of this nature may be capable of detecting one, two, three, four, five, six, seven, eight, nine or ten different analyte types during operation.

In another form of this assay, a known amount of a given analyte may be bound to a signalling molecule, and the device may comprise an immobilised detection molecule population (e.g. antibodies and/or aptamers) with binding specificity for a ligand bound to the analyte. Various populations of different immobilised detection molecule populations with binding specificity for different types of ligands may be present and arranged in a spatially separated manner. Upon application to the device, the known amount of analyte bound to the signalling molecule competes for binding to the detection molecule population with an unknown amount of the same analyte that is not bound to the signalling molecule. The positive control may be indicative of binding of the analyte (of known amount) bound to the signalling molecule to the immobilised detection molecule population. A competitive format of this nature may be capable of detecting one, two, three, four, five, six, seven, eight, nine or ten different analyte types during operation.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLE(S)

The present invention will now be described with reference to specific example(s), which should not be construed as in any way limiting.

### Example 1

### Multiplex Lateral Flow Detection and Binary Encoding Enables a Molecular Colourimetric 7-Segment Display

Introduction

Multiplexing is a critical parameter for increasing diagnostic efficiency. The strategies that enable simultaneous analysis of multiple samples are largely dependent on the underlying diagnostic technology. Mature technologies such as enzyme-linked immunosorbent assay (ELISA) and real-time polymerase chain reaction (PCR) enable limited multiplexing in laboratory and clinical settings, but are time-consuming to perform. Contemporary methods focus on microarray-based technologies coupled with nanomaterials (e.g. magnetic nanoparticles) for detection, such as the Bio-Rad Bio-Plex® Systems and Luminex MagPix®. These enable reduced sample volume and shorter detection times using high-throughput and even automated processing methods. However, all of these multiplexing technologies require highly equipped institutions with skilled technicians for operation and, therefore, preclude deployment to low resource or point-of-care settings.

Lateral flow devices (LFDs) are ideal candidates for low resource and point-of-care implementation due to their rapidness, simplicity and low cost, whilst retaining high diagnostic sensitivity and specificity. However, very few commercially available LFDs employ multiplexing. The most direct LFD multiplexing strategy is to simply increase the number of test lines along the length on a single device. However, such linear expansion is limited, as described by Washburn's theory that the flow rate in a porous matrix is inversely proportional to the wicking distance. Thus, the flow rate decreases with distance from the conjugate pad and consequently the assay time increases. In addition, the number of test lines that can be added is limited by the size of the device. Alternatively, some commercial LFDs accommodate several parallel dipsticks in a single cassette, such as the BD™ Directigen™ EZ Flu A+B kit (Franklin Lakes, N.J.), the Alere BinaxNOW® Influenza A&B Card (Orlando, Fla.) and the RAID 8 and RAID TOX (Wheeling, Ill.). This does not directly address multiplex expansion, as it increases reagent consumption in multiples of the single dipstick. Apart from parallelism, others have increased multiplexing in a multi-direction manner, providing more arms or zones to accommodate more test lines. However, this strategy consumes as much reagent as the parallelism strategy and increases the dimension size of the devices.

In the present Example a novel solution is proposed for expanding multiplex detection using a lateral flow system that increases the efficiency of detection without consuming excess reagents. The solution compresses multiplex LFD data by borrowing from computational science and uses binary encoding to create signature patterns of test dots. Thus, a sample is diagnosed based on the set of test dots that appear on the device. Moreover, by judicious arrangement of test dots facilitates simulation of digital displays that generate alphanumeric outputs. To demonstrate, a standard sandwich immunoassay with gold nanoparticles (AuNPs) as signal transducer was employed and seven antigen-antibody pairs that operate reliably for hepta-plex detection on a linear scale were identified. A 7-segment display format was developed such that addition of specific label signatures resulted in the appearance of unique numeric codes, generating the numbers 0-9 on the display.

The system out-performs other colourimetric multiplex LFDs that detect via hapten-antibody reactions. It allows detection of up to seven entities simultaneously on a single strip (compared to the penta-plex lateral flow detection previously reported). In addition, by employing binary encoding, a completely new mechanism is provided that enables the diagnosis of 10 unique entities using only seven antigen-antibody pairs. Theoretically, the hepta-plex system could be used to decipher 127 (2\hat{0}7-1) entities in a single LFD device, indicating the power of binary and molecular encoding for the development of compact multiplex POC detection systems. Moreover, the 7-segment display demonstrates, for the first time, a digital-like alphanumeric display within a LFD. This pre-programmed readable text is ideal for POC diagnosis in low resource settings, as it does not require electricity but is powered entirely by molecular interactions.

Materials and Methods

Oligonucleotides and Antibodies

Nucleic acid lateral flow detection was performed on single-stranded synthetic dual-labelled DNA encoding a segment of the Rift Valley Fever virus (RVFV) L gene (5′ tgctaggctaagaccagtaagcaaagtcaggcttagatttaggga 3′ (Genbank accession number NC_014397.1, nucleotides 6287-6331). This same sequence was used to generate all twelve dual-labelled DNA products to ensure consistency and preclude that individual differences in binding would not be due to subtle changes in DNA sequence. DNA was dual labelled with 6-carboxyfluorescein (6-FAM) at the 5′ end, and at the 3′ end with either Biotin, CY5Sp, Digoxigenin N, 6-TAMRASp or Texas Red-XN, and synthesised and HPLC purified by Integrated DNA Technologies, Inc (IDT., Coralville, USA). Similarly, dual-labelled DNA with a 3′ end of either Alexa 488 C6-NH, BODIPY FL C5 C6-NH, Cascade Blue C6-NH, DNP-X C6-NH or Dansyl-X C6-NH were synthesised by TriLink BioTechnologies (TriLink BioTechnologies, San Diego, USA) and PAGE purified. Finally, dual-labelled DNA with a 3′ end of either Lucifer Yellow or Benzopyrene were synthesised by Bio-Synthesis (Bio-Synthesis, Inc., Lewisville, USA) and dual HPLC purified.

A monoclonal anti-fluorescein antibody (Roche Diagnostics Australia Pty. Ltd., Castle Hill, NSW, Australia) was used for AuNP conjugation. Antibodies or ligands corresponding to the 3′-labelled DNA were: (i) streptavidin (New England Biolabs, Arundel, QLD, Australia); (ii) monoclonal anti-Cy5 antibody (Sapphire Bioscience Pty. Ltd., Waterloo, NSW, Australia); (iii) polyclonal anti-Digoxigenin antibody (Roche Diagnostics Australia Pty. Ltd., Castle Hill, NSW, Australia); (iv) monoclonal anti-TAMRA antibody (Thermo Fisher Scientific, Scoresby, VIC, Australia); (v) monoclonal anti-Texas Red antibody (Invitrogen Corporation, Carlsbad, Calif., USA); (vi) polyclonal s anti-Alexa Fluor® 488 antibody (Life Technologies Australia Pty Ltd., Mulgrave, VIC, Australia); (vii) polyclonal anti-BODIPY® FL antibody (Life Technologies Australia Pty Ltd., Mulgrave, VIC, Australia); (viii) polyclonal anti-Alexa Fluor® 405/Cascade Blue® antibody (Life Technologies Australia Pty Ltd., Mulgrave, VIC, Australia); (ix) polyclonal anti-Dinitrophenyl-KLH antibody (Life Technologies Australia Pty Ltd., Mulgrave, VIC, Australia); (x) polyclonal anti-Dansyl antibody (Life Technologies Australia Pty Ltd., Mulgrave, VIC, Australia); (xi) polyclonal Lucifer Yellow antibody (Life Technologies Australia Pty Ltd., Mulgrave, VIC, Australia); (xii) and monoclonal anti-Benzo(a)pyrene antibody (Biorbyt Ltd., Cambridge, Cambridgeshire, United Kingdom). These antibodies were deposited at the test zone of the nitrocellulose membrane. In addition, a polyclonal rabbit anti-mouse antibody (Sapphire Bioscience Pty. Ltd., Waterloo, NSW, Australia) was deposited at the control zone.

Preparation of GNPs Conjugates

Anti-fluorescein antibody was coupled to AuNPs, which served as the signaling molecule (red in colour) to allow the visualisation of the immuno-sandwich complex by eye. Coupling to AuNPs (40 nm, 20 OD) was performed using the InnovaCoat® GOLD 10× Multi Explorer labelling kit (BioNovus Life Sciences, Cherrybrook, NSW, Australia). Briefly, reagents were thawed to 25° C., and 12 μL antibody (diluted to 0.1 mg/mL using the diluent provided) was mixed with 42 μL reaction buffer. The mixture (45 μL) was used to resuspend a vial of InnovaCoat GOLD nanoparticles, which was incubated for 10 min before addition of 5 μL Quencher, resulting in a final 20 OD solution (50 μL) of anti-fluorescein/AuNP. The conjugates were washed twice with the borate running buffer by centrifuging at 7000×g for 6 min, before resuspension to the original (50 μL) volume. Conjugates were stored at 4° C.

Preparation of Single-Plex Nucleic Acid Lateral Flow Strip and Multiplexed Nucleic Acid Lateral Flow Strip

Conjugate and sample pads (Millipore, Billerica, Mass., USA) were blocked with blocking solution (1% polyvinyl alcohol, 20 mM Tris base, pH 7.4) for 30 min and dried at room temperature for 2 h. The two pads were then impregnated in borate running buffer (100 mM H₃BO₃, 100 mM Na₂B₄O7, 1% BSA, 0.05% Tween 20, pH 8.8) by soaking in buffer for 30 min before drying at 25° C. overnight.

Assembled devices (6.1 cm×0.3 cm) for single-plex lateral flow detection comprised treated sample pad (1.5 cm), treated conjugate pad (0.6 cm), a nitrocellulose membrane (2.5 cm; Hi-Flow Plus HF135) and an absorbent pad (1.5 cm) (Millipore, Billerica, Mass., USA) combined on an adhesive backing card (Lohmann Corporation, Hebron, Ky., USA), with a 0.1 cm overlap between components. The multiplexed lateral flow dipsticks were assembled using the same components and procedure, using treated sample pad (0.5 cm), treated conjugate pad (0.6 cm), a nitrocellulose membrane (3.5 cm) and an absorbent pad (2.5 cm).

For single-plex lateral flow detection, detection ligand or antibodies [either streptavidin (1.0 mg/mL), anti-Cy5 antibody (2.0 mg/mL), anti-Digoxiginin (0.75 U/μL), anti-TAMRA (1.0 mg/mL), anti-Texas Red antibody (1.0 mg/mL), anti-Alexa Fluor® 488 (1.0 mg/mL), anti-BODIPY® FL (3.0 mg/mL), anti-Alexa Fluor® 405/Cascade Blue® (3.0 mg/mL), anti-Dinitrophenyl-KLH (2.0 mg/mL), anti-Dansyl (1.0 mg/mL), anti-Lucifer Yellow (3.0 mg/mL) or anti-Benzo(a)pyrene (1.0 mg/mL)] were pipetted (0.4 μL) onto the test zone of the nitrocellulose membrane. Rabbit anti-mouse antibody (1 mg/mL in 50% glycerol) was pipetted (0.4 μL) at the control zone. Test and control antibodies were spotted 0.5 cm apart and dried at 25° C. for 45 min. For multiplexed lateral flow detection, twelve or seven detection ligands or antibodies (0.2 μL) were deposited and as a control, rabbit anti-mouse antibody was pipetted (0.2 μL) in triplicate at the end of each array.

Single-Plex Lateral Flow Test and Multiplexed Lateral Flow Test Procedure

Single-plex lateral flow detection was performed as described previously¹². Briefly, anti-fluorescein/AuNP conjugate (1 μL) was pipetted onto the conjugate pad and the strip was dipped into a mixture containing 100 μL running buffer and 1 μL dual-labelled RVFV DNA (1 to 0.0005 μM). An additional 1 μL anti-fluorescein/AuNP conjugate was pipetted onto the conjugate pad once the running buffer reached the bottom of the absorbent pad as this double-run method has been demonstrated to be effective for developing high signal intensity with reduced anti-fluorescein/AuNP consumption. The strip was developed for 15 min. Negative control strips (no DNA) were run in parallel. All experiments were performed in duplicate and repeated at least twice to demonstrate consistency of results.

For the multiplexed lateral flow detection, the strip was run using mixtures of single-stranded synthetic RVFV DNAs (1 μM; 0.3 μL of 5′FAM/3′Benzopyrene, 5′6-FAM/3′CY5Sp, 5′6-FAM/3′Digoxigenin_N, and 5′FAM/3′ DNP-X C6-NH; 0.4 μL of 5′FAM/3′Alexa 488 C6-NH, 5′6-FAM/3′Biotin and 5′6-FAM/3′BODIPY FL C5 C6-NH; 0.5 μL of 5′FAM/3′ Cascade Blue C6-NH, 5′6-FAM/3′6-TAMRASp and 5′6-FAM/3′Texas Red-XN; 0.6 μL of 5′FAM/3′Dansyl-X C6-NH; 1.0 μL 5′FAM/3′Lucifer Yellow) and 150 μL running buffer firstly, and then anti-fluorescein/AuNP conjugate (5 μL) were pipetted onto the conjugate pad and finished developing with another 100 μL s running buffer for 25 min. All experiments were repeated at least three times.

7-Segment Lateral Flow Test Procedure

The 7-segment display of numbers 0-9 were tested by adding different dual-labelled DNA mixtures (out of seven: 5′6-FAM/3′Digoxigenin_N, 5′6-FAM/3′Texas Red-XN, 5′6-FAM/3′6-TAMRASp, 5′FAM/3′ Cascade Blue C6-NH, 5′FAM/3′ DNP-X C6-NH, 5′6-FAM/3′Biotin and/or 5′FAM/3′Dansyl-X C6-NH) with the same concentration and amount mentioned above) in the borate running buffer:

Number “0”: 5′6-FAM/3′Digoxigenin_N, 5′6-FAM/3′Texas Red-XN, 5′6-FAM/3′6-TAMRASp, 5′FAM/3′ DNP-X C6-NH, 5′6-FAM/3′Biotin and 5′FAM/3′Dansyl-X C6-NH.

Number “1”: 5′6-FAM/3′6-TAMRASp and 5′6-FAM/3′Biotin.

Number “2”: 5′6-FAM/3′Digoxigenin_N, 5′6-FAM/3′6-TAMRASp, 5′FAM/3′Cascade Blue C6-NH, 5′FAM/3′ DNP-X C6-NH and 5′FAM/3′Dansyl-X C6-NH.

Number “3”: 5′6-FAM/3′Digoxigenin_N, 5′6-FAM/3′6-TAMRASp, 5′FAM/3′ Cascade Blue C6-NH, 5′6-FAM/3′Biotin and 5′FAM/3′Dansyl-X C6-NH.

Number “4”: 5′6-FAM/3′ Texas Red-XN, 5′6-FAM/3′6-TAMRASp, 5′FAM/3′ Cascade Blue C6-NH and 5′6-FAM/3′Biotin.

Number “5”: 5′6-FAM/3′Digoxigenin_N, 5′6-FAM/3′ Texas Red-XN, 5′FAM/3′ Cascade Blue C6-NH, 5′6-FAM/3′Biotin and 5′FAM/3′Dansyl-X C6-NH.

Number “6”: 5′6-FAM/3′Digoxigenin_N, 5′6-FAM/3′ Texas Red-XN, 5′FAM/3′ Cascade Blue C6-NH, 5′6-FAM/3′Biotin, 5′FAM/3′ DNP-X C6-NH and 5′FAM/3′Dansyl-X C6-NH.

Number “7”: 5′6-FAM/3′Digoxigenin_N, 5′6-FAM/3′6-TAMRASp and 5′6-FAM/3′Biotin.

Number “8”: 5′6-FAM/3′Digoxigenin_N, 5′6-FAM/3′ Texas Red-XN, 5′6-FAM/3′6-TAMRASp, 5′FAM/3′ Cascade Blue C6-NH, 5′FAM/3′ DNP-X C6-NH, 5′6-FAM/3′Biotin and 5′FAM/3′Dansyl-X C6-NH.

Number “9”: 5′ 6-FAM/3′ Digoxigenin_N, 5′6-FAM/3′ Texas Red-XN, 5′6-FAM/3′6-TAMRASp, 5′FAM/3′ Cascade Blue C6-NH, 5′6-FAM/3′ Biotin and 5′FAM/3′Dansyl-X C6-NH.

All the seven corresponding antibodies were pre-deposited on the nitrocellulose membrane. The running procedure of the multiplexed lateral flow test was performed as for the 7-segment lateral flow test.

Image Analysis

Reacted lateral flow dipsticks were dried, imaged using the MultiDoc-ItTM Digital Imaging System (Upland, Calif., USA), and analyzed using ImageJ software (National Institutes of Health, Md., USA). Image brightness/contrast and colour balance were auto-adjusted. The background was subtracted against negative-control dipsticks and the spot intensity was reported as mean gray value. The lowest detection limits were determined by the test dot intensity values that were below the average plus three standard deviations. Statistical differences between test dot intensities were analysed by t-test using PRISM (Graphpad software Inc. version 6.0 Mac, San Diego, California).

### Results

Single-Plex Nucleic Acid Lateral Flow Testing

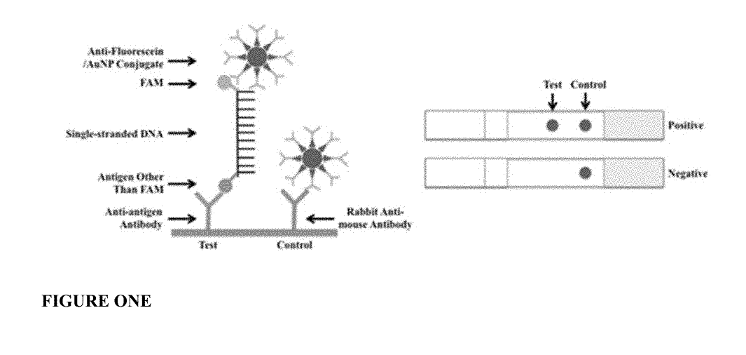

In order to develop a multiplex LFD, twelve individual single-plex assays were developed using a sandwich immunoassay format. In this format an analyte is sandwiched between a capture and detection antibody via tagged antigens (see FIG. 1 which shows design parameters for single-plex LFDs).

FIG. 1: A dual-labelled analyte (RVFV DNA) is sandwiched between capture and detection antibodies due to hapten/antibody binding (FIG. 1, left panel). The tracer, AuNPs conjugated to the capture antibody (mouse anti fluorescein antibody), enables visualisation of binding due to the appearance of a red color at the test dot (FIG. 1, right panel). A control reaction is achieved by depositing rabbit anti-mouse antibody, which can directly bind the capture antibody (mouse anti-fluorescein). This validates the assay as the signal appears even in the absence of analyte.

Thus, AuNPs conjugated to the capture antibody transforms the sandwich complex (capture antibody/AuNPs/nucleic acid/detection antibody) into a colourimetric signal. Fluorescein and anti-fluorescein was chosen as the common AuNP conjugated capture antibody.

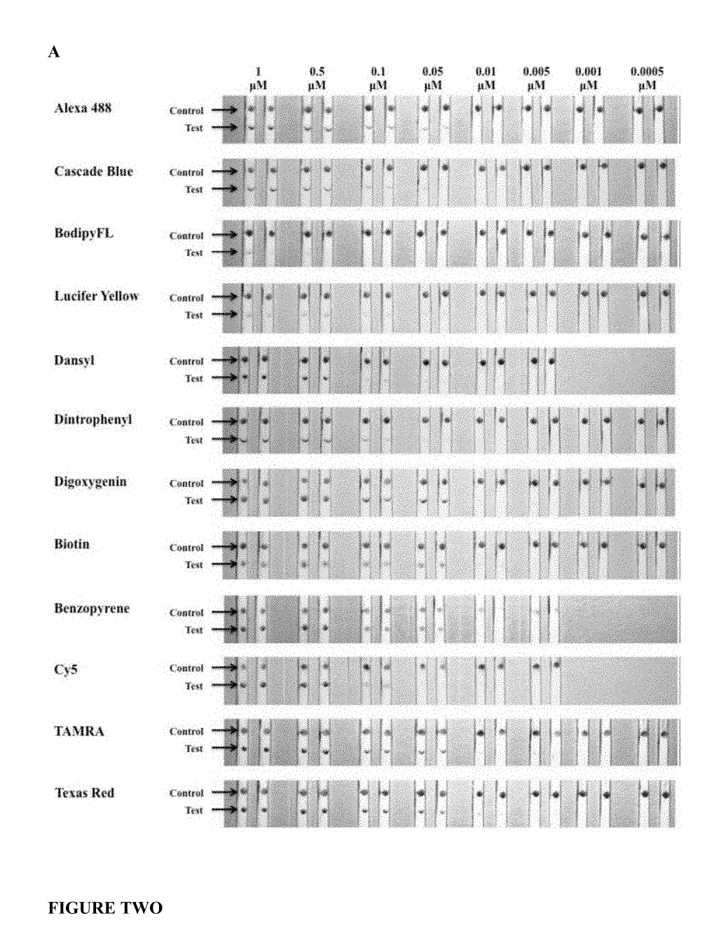

An additional twelve commercially available antigen-antibody combinations were identified that could potentially be used as detection antibodies (FIG. 2). The test analyte was dual-labelled single-stranded DNA corresponding to a segment S gene portion of Rift Valley Fever virus.

FIG. 2A. Single-plex lateral flow detection results for twelve antigen-antibody pairs. Detection antibody (0.4 μL) corresponding to each antigen was deposited on the nitrocellulose membrane and dried at room temperature for 45 min. Control rabbit anti-mouse antibody (0.4 μL) was similarly added 0.5 cm above each test dot for assay validation. Each strip was tested using a pre-developed double-run method, which involves dipping the lateral flow dipstick into a mixture containing 100 μL running buffer and 1 μL dual-labelled analyte (RVFV DNA), followed by pipetting an additional 1 μL anti-fluorescein/AuNP conjugate onto the conjugate pad once the running buffer reached the bottom of the absorbent pad. Analyte concentrations tested were from 1 to 0.0005 μM. The strip was developed for 15 min. Reacted lateral flow strips were dried overnight, imaged using the MultiDoc-ItTM Digital Imaging System, and analysed using Image) software. Labels Alexa488, Cascade Blue, Lucifer Yellow, Benzopyrene, BodipyFL, and Dansyl, which have not previously been applied in lateral flow detection. Antigen-antibody pairs Digoxigenin/anti-Digoxigenin, TAMRA/anti-TAMRA, and Texas Red/anti-Texas Red had the lowest detection limits (0.005 μM of DNA), while Cy5/anti-Cy5 (0.1 μM of DNA), and BodipyFL/anti-BODIPY® FL (0.5 μM of DNA) pairs had the highest detection limits. Interestingly, the dot morphology and intensity varied among different antigen-antibody pairs. Only Biotin/Streptavidin, Benzopyrene/anti-Benzo(a)pyrene, and Cy5/anti-Cy5 pairs did not show a “coffee ring effect” as the DNA concentration decreased. However, this effect was different to that described previously where it has been suggested the uneven staining is due to capillary flow from different evaporation rates across the drop (where liquid evaporating from the edge is replenished by liquid from the interior). The “coffee ring effect” observed in the present Example was dependent on the analyte (DNA concentration), and this phenomenon has not previously been reported. Additionally, the label pairs Biotin/Streptavidin, Lucifer Yellow/anti-Lucifer Yellow, and BodipyFL/anti-BODIPY® FL showed less intense test dots compared to the other pairs.

FIG. 2B: shows the lowest detection limits of each antigen-antibody pair in the single-plex lateral flow detection. Colour intensity (quantitated using Image) software) is plotted against DNA concentration with data points and error bars indicating the average and standard deviation of 4 individual tests. The solid black line represents the cut-off used to determine the lowest concentration at which a signal could still be detected (defined as three standard deviations above the average negative values).

All twelve antigen-antibody pairs were effective as detection entities in the single-plex LFDs (FIG. 2), and in particular the success of labels Alexa488, Cascade Blue, Lucifer Yellow, Benzopyrene, BodipyFL and Dansyl were noted, which have not previously been applied in lateral flow detection. Antigen-antibody pairs Digoxigenin/anti-Digoxigenin, TAMRA/anti-TAMRA and Texas Red/anti-Texas Red had the lowest detection limits (0.005 μM of DNA), while Cy5/anti-Cy5 (0.1 μM of DNA) and BodipyFL/anti-BODIPY® FL (0.5 μM of DNA) pairs had the highest detection limits (FIG. 2). Interestingly, the dot morphology and intensity varied among different antigen-antibody pairs. Only Biotin/Streptavidin, Benzopyrene/anti-Benzo(a)pyrene and Cy⁵/anti-Cy5 pairs did not show a “coffee ring effect” as the DNA concentration decreased. However, this effect was different to that previously described where it was suggested the uneven staining is due to capillary flow from different evaporation rates across the drop (where liquid evaporating from the edge is replenished by liquid from the interior). The “coffee ring effect” observed was dependent on the analyte (DNA concentration), and this phenomenon has not previously been reported. Additionally, the label pairs Biotin/Streptavidin, Lucifer Yellow/anti-Lucifer Yellow and BodipyFL/anti-BODIPY® FL showed less intense test dots compared to the other pairs.

Multiplex Nucleic Acid Lateral Flow Testing

After demonstrating that all twelve detection antigen-antibody combinations successfully operated in a single-plex LFD, it was attempted to combine these into a multiplex LFD array using a dot-matrix (3×4) format. For the arrangement of detection antibodies on the membrane, it was hypothesised that the antibodies furthest from the conjugate pad would suffer the most from loss of reagents, and thus the most logical arrangement of detection antibodies would be to place the least sensitive detection antibodies closest to the conjugate pad. Importantly, the RVFV DNA analyte sequence was kept identical in all tests (apart from the antigen labels) to eliminate any differences in behavior due to cross-reacting nucleotide sequences.

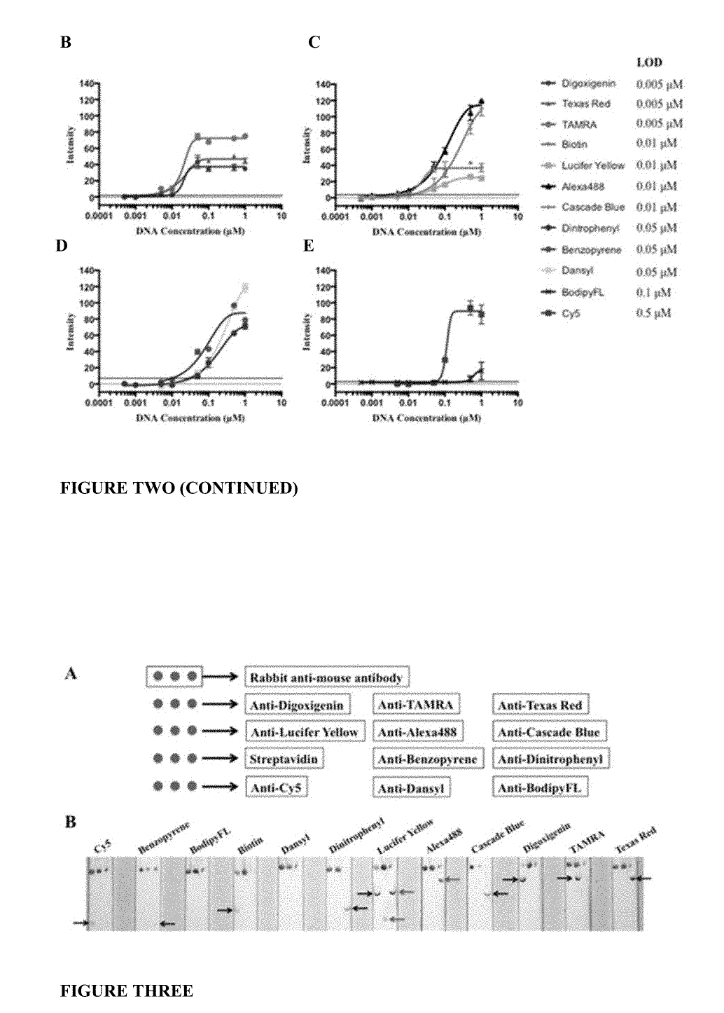

One concern was whether the antigens would non-specifically bind to other detection antibodies, and thus it was first tested the specificity of the twelve antigen-antibody pairs on a multiplex LFD 3×4 array. The results indicated three non-specific antigen/detection antibody reactions: (1) Benzopyrene bound non-specifically with anti-BODIPY® FL antibody; (2) Lucifer Yellow bound non-specifically with anti-Alexa Fluor® 405/Cascade Blue® and anti-Dansyl antibodies; and (3) Alexa488 bound non-specifically with anti-Texas Red antibody (FIG. 3). Curiously, the Alexa488 bound only to the anti-Texas Red antibody and not to its corresponding anti-Alexa Fluor® 488 antibody in this particular configuration (FIG. 3).

FIG. 3: Specificity test results of all the twelve antigen-detection antibody pairs. A: positioning of the detection antibodies in the multiplex LFD 3×4 array. B: Dual-labelled DNA sample containing FAM/X (where X=Cy5, Benzopyrene, BodipyFL, Biotin, Dansyl, Dinitrophenyl, Lucifer Yellow, Alexa488, Cascade Blue, Digoxigenin, TAMRA or Texas Red) was added as shown. The black arrow denotes the correct test dot while the red arrow denotes the incorrect test dot. The assay was independently performed three times with similar results and a representative photograph from one test is shown.

The BodipyFL and Dansyl antigen-antibody pairs did not produce test dots in the 3×4 LFD array. Two possibilities were considered for this lack of binding: (1) the antigen-antibody pairs with low sensitivity become even less sensitive when tested in a lateral flow dipstick that has a larger surface area; and/or (2) presence of other detection antibodies interfere with the binding of BodipyFL and Dansyl antigens to their corresponding detection antibodies. To test these possibilities, the LFD was reduced to a 7-dot array containing six detection antibodies chosen due to their superior specificity (see FIG. 3), with a seventh detection antibody of either anti-BodipyFL, anti-Dansyl or anti-Cy5 detection antibody.

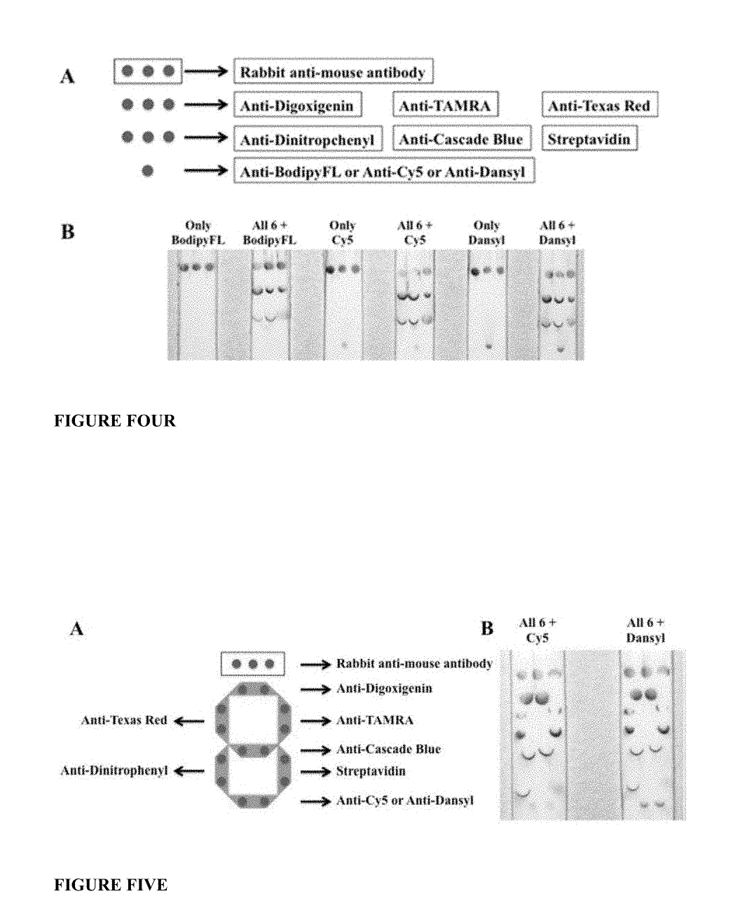

FIG. 4: Hepta-plex lateral flow detection results for the combination of potential compatible seven antigen-antibody pairs for the 7-segment display of numbers. A: positions of the antigen-antibody pairs upon the lateral flow stick. B: hepta-plex lateral flow detection results of combining Biotin, Cascade Blue, Digoxigenin, Dintrophenyl, TAMRA and Texas Red to either BodipyFL, Cy5 or Dansyl antigen-antibody pairs. Representative photograph from at least three repeated assays (with all DNA concentrations at 1 μM).

Anti-Cy5 was included as a comparative control. Testing was performed either using dual-labelled analyte containing either Bodipy, Dansyl or Cy5 antigens or with an analyte reaction mix containing all seven antigens corresponding to the seven detection antibodies present on the strip. Interestingly, the BodipyFL antigen still did not produce any observable test dots, whereas the Dansyl test dots appeared and showed specificity in the seven antigen-antibody pairs system (FIG. 4B). The lack of Dansyl binding in the 3×4 array but binding in the 7-dot array was consistent in all tests and supports a second theory: the presence of some detection antibodies precludes Dansyl binding despite it being placed in the first row of the 3×4 array. In contrast, the lack of BodipyFL binding is most likely to be due to the larger surface area ablating detection, since BodibyFL is the least sensitive in the single-plex assays (FIG. 2).

7-Segment Display and Binary Encoding

On a linear scale, the 7-dot LFD array is an improvement on the current maximum multiplex detection that employs hapten-antibody reactions, since only penta-plex LFD has been previously reported. However, stacking the test lines (or dots) along the flow path of the lateral flow device does not provide an intuitive result output, so an alternative approach was considered to present the information. The identified 7-label system was applied to display numbers by arranging them in a dot matrix format, analogous to a 7-segment display.

To demonstrate this proof-of-concept, a 7-dot array was chosen that incorporated Dansyl, since the Dansyl antigen-antibody pair demonstrated significant relative higher dot intensity (p<0.05) compared to the Cy5 antigen-antibody pair in both the single-plex and 7-dot LFD tests (FIGS. 2 and 4). Each detection antibody was used to represent one segment of the display. As with the 3×4 and 7-dot LFD arrays, the positioning of the seven detection antibodies was in ascending sensitivity order corresponding to distance from the conjugate pad. Duplicate test dots were initially used to represent each segment (FIG. 5A), however, this resulted in position effects where the morphology of the second test dot was influenced by the first test dot in the vertical segments (FIG. 5B).

FIG. 5. 7-segment display results. A: Positioning of the detection antibodies, where each segment of the display was represented by duplicate detection antibody deposition to create two test dots. B: The numerical number “8” appeared by the addition of analytes labelled with a molecular antigen signature consisting of Biotin, Cascade Blue, Digoxigenin, Dintrophenyl, TAMRA, and Texas Red combined with either Cy5 or Dansyl.

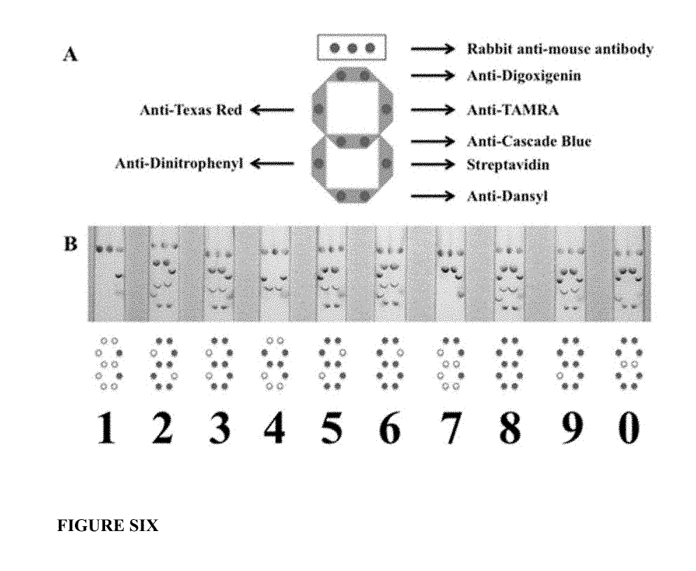

Therefore, the vertical segments were reduced to only incorporate one test dot (FIG. 6A). The deposition position of the detection antibodies defined the unique molecular signature required to make meaningful outputs on the display. In this way mixtures of labelled analyte were created to display each number 0-9 and tested them for performance (FIG. 6B). Results consistently showed that clear numbers 0-9 could be generated on a lateral flow device using the novel 7-segment LFD display design (FIG. 6B).

FIG. 6. 7-segment display of numbers on lateral flow dipsticks. A: Positioning of the detection antibodies, which forms the 7-segments of the display. B: Addition of labelled analyte signature mixtures (with all DNA concentrations at 1 μM) resulted in the successful appearance of numbers (0 to 9). The assay was performed three times with similar results and a photograph of one test is shown.

The successful demonstration of the numbers 0-9 using seven antigen-antibody pairs on a LFD is the first digital-like display of numbers on a paper-based biosensor. It operates as a single-use feed-forward circuit that employs a pre-defined molecular encoding strategy for information transfer. The current detection technology has advantages that are particularly important for point-of-care diagnosis, and include: (1) decreased detection time (20 min versus 60 min); (2) visualisation of signal by eye inspection (rather than fluorescent signal); and (3) only a single addition of sample is required (rather than requiring sample to be added discretely to each segment of the display).

The concept of multiplexing in lateral flow devices has been previously explored and includes the use of multiple lines (with the same or different colours) on a single device, using micro-array style matrices; as well as bi-directional, parallel or multi-directional systems. Expansion of multiplex detection of these systems is hampered by either space limitations within the original device dimension or the requirement to consume more reagents as the device size increases. The most compact system is the microarray style matrix of dots, which minimises both reagent consumption and device dimension. However, the read-out from the devices is non-intuitive, imposing difficulties in interpreting results. Previously demonstrated intuitive read-outs have displayed blood types on a paper-based biosensor using letters deposited in different sections of the biosensor (not overlayed), each image being created from the deposition of a single antibody. Thus expansion for the detection of more analytes would require both additional reagents and space on the display.

The current technology provides a novel solution to enable compaction of multiplexing by borrowing from computational science and employing a binary encoding scheme that moves beyond space limitations. The computational strategy employed differs from demonstrations that showcase embedded molecular logic gates on paper-based biosensors. The present method involves strategic manipulation of the inputs to include permutation and combinations of labels such that each input incurs a unique pattern. Noticeably, this binary encoding scheme only applies to differentiation of discrete analytes and different encoding schemes would need to be employed to differentiate analyte mixtures. However, with the identified seven labels, it is possible to theoretically detect 127 (2\hat{0}7-1) discrete analytes on a single device. This number would likely be reduced in practice to incorporate redundancy for error detection during read-out. In this paper the outputs were intentionally restricted to a numerical system to demonstrate multiplex analyte detection while simultaneously providing an intuitive read-out applicable to point-of-care diagnostics.

The present displays are generic and can be applied for multiplex detection of any candidates (e.g. nucleic acids, protein, lipid or small molecules) that are incorporated with the specific recognition labels. In addition, this lateral flow detection technology is amenable to upstream nucleic acid amplification, where the specific recognition labels can be incorporated into the analyte(s) using primers and probes. This would additionally assist with detection of low copy numbers.

### Conclusions

In this study, the multiplexing of lateral flow devices was improved to a hepta-plex detection on a linear scale. An intuitive result output was also developed—a 7-segment display of numbers on a paper-based biosensor. Further multiplexing of the device can be achieved using the binary encoding concept, and this allows theoretical detection of up to 127 different entities using only seven label pairs. This demonstration is an innovation in the development of compact molecular biosensors using an information encoding strategy. Such an approach is a step towards developing cost-effective and personalised point-of-care diagnostics.

### Example 2

### Multiplex Nucleic Acid Lateral Flow Detection and Binary Encoding Enables a Molecular Colourimetric 7-Segment Display

Introduction

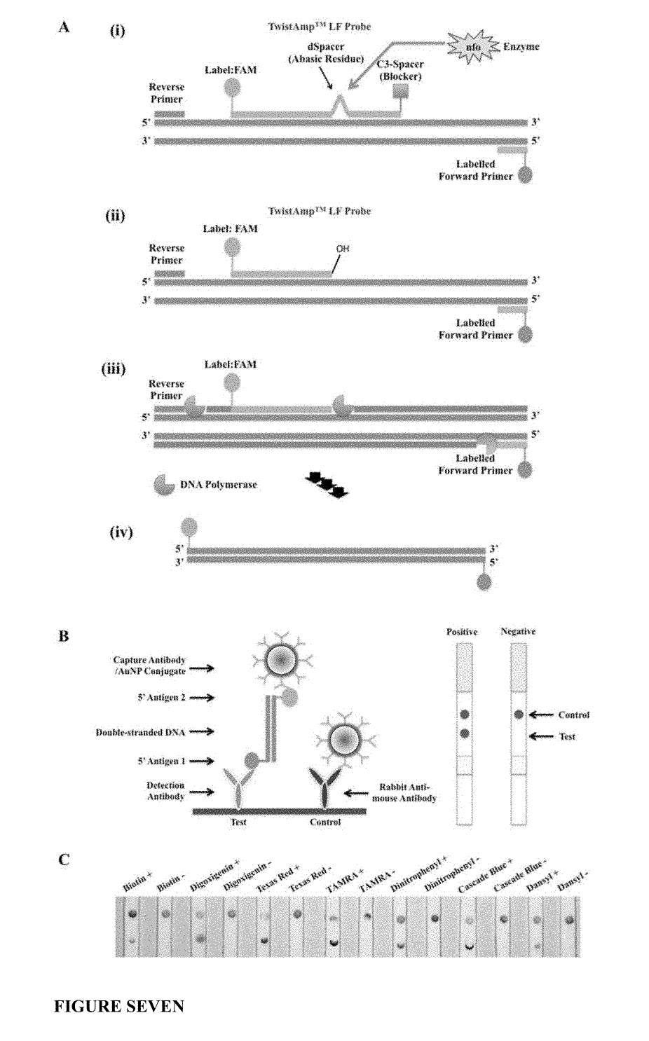

Nucleic acid amplification is a critical tool in diagnostics and integration with isothermal amplification and nucleic acid lateral flow (NALF) extends applicability of the technology towards low-resource, point-of-care (POC) applications. Detection without the use of electronic devices is enabled through incorporation of 5′ primer labelling during amplification and subsequent reaction with label detection technologies embedded within the NALF strip¹³ (FIG. 7). However, multiplexed detection using these technologies is problematic because (1) the chemical structure of commercially available 5′ primer labels may not be compatible with the isothermal amplification system, (2) there is a physical limit to detection expansion of a lateral flow device (LFD) because flow rate decreases with distance, and (3) user interpretation becomes difficult as the number of lines or dots accumulate, necessitating incorporation of electronic reading devices which reduces low-resource applicability. To circumvent these issues, the present inventors have provided a novel LFD technology that combines binary and molecular encoding to increase multiplex detection capacity without expanding device dimensions (see Example 1 above). This platform provides an intuitive result readout as a 7-segment display of numbers on the LFDs. Here the multiplexing potential for low-resource POC applications is demonstrated by combining this colourimetric 7-segment display technology with an isothermal nucleic acid amplification strategy.

Materials and Methods

Oligonucleotides and Antibodies

DNA template, RPA primers (5′ labelled with Biotin, Digoxigenin (NHS Ester), TAMRA™ (NHS Ester) or Texas Red®-X (NHS Ester)) and probe (Table 8) were synthesised and HPLC purified by Integrated DNA Technologies, Inc (IDT., Coralville, USA). The RPA primer 5′ labelled with Cascade Blue C6-NH was synthesised and HPLC purified by Invitrogen (Life Technologies Australia Pty Ltd., Mulgrave, VIC, Australia). The RPA primers 5′ labelled with DNP-X C6-NH or Dansyl-X C6-NH were synthesised by TriLink BioTechnologies (TriLink BioTechnologies, San Diego, USA) and PAGE purified.

A monoclonal anti-fluorescein antibody (Roche Diagnostics Australia Pty. Ltd., Castle Hill, NSW, Australia) was used for gold nanoparticles (AuNPs) conjugation. Antibodies corresponding to the 5′-labelled DNA were: (i) anti-biotin (mouse) monoclonal antibody (Rockland Immunochemicals Inc., Limerick, Pa., USA); (ii) polyclonal anti-digoxigenin antibody (Roche Diagnostics Australia Pty. Ltd., Castle Hill, NSW, Australia); (iii) monoclonal anti-TAMRA antibody (Thermo Fisher Scientific, Scoresby, VIC, Australia); (iv) monoclonal anti-Texas Red antibody (Invitrogen Corporation, Carlsbad, Calif., USA); (v) polyclonal anti-Alexa Fluor® 405/Cascade Blue® antibody (Life Technologies Australia Pty Ltd., Mulgrave, VIC, Australia); (vi) polyclonal anti-Dinitrophenyl-KLH antibody (Life Technologies Australia Pty Ltd., Mulgrave, VIC, Australia); and (vii) polyclonal anti-Dansyl antibody (Life Technologies Australia Pty Ltd., Mulgrave, VIC, Australia). These antibodies were deposited at the test zone of the lateral flow strip. In addition, a polyclonal rabbit anti-mouse antibody (Sapphire Bioscience Pty. Ltd., Waterloo, NSW, Australia) was deposited at the control zone.

RPA Amplification

RPA amplification was performed using synthetic double-stranded DNA encoding a segment of the Rift Valley fever virus S gene (Genbank accession number NC_014395.1, nucleotides 1428-1535)[1], a pair of forward (5′ labelled with either Biotin, Digoxigenin (NHS Ester), TAMRA™ (NHS Ester), Texas Red®-X (NHS Ester), Cascade Blue C6-NH, DNP-X C6-NH or Dansyl-X C6-NH) and reverse primers, and a Nfo probe (5′ labelled with 6-carboxyfluorescein (FAM), containing an internal dSpacer replacing a base, and a 3′ C3-Spacer carbon blocker) (Table 8).

TABLE 8
Sequences of primers, probe and DNA template for
RPA amplification

| Name | Sequence (5′ to 3′) |
|---|---|
| RVFV-S-F | X-CATTTTCATCATCATCCTCCKGGGSTTRTTG |
| RVFV-S-R | GARCTCYTAAAGCAGTATGGTGGGGCTGACT |
| RVFV-S- | FAM-GGGAGAAGGATGCCAAGAAAATGATTGTT |
| Nfo-Probe | (dSpacer)TGGCTCTRACTCGTG(C3-Spacer) |
| RVFV-DNA | GCTTTGCCTTCTTTCGACATTTTCATCATCATCCTCCT |
| | GGGCTTGTTGCCACGAGTTAGAGCCAGAACAATCATTT |
| | TCTTGGCATCCTTCTCCCAGTCAGCCCCACCATACTGC |
| | TTTAAGAGTTCGATAACTCTACGGGCATCAAACCC |

Single-Plex RPA Amplification

The RPA reaction was conducted at 39° C. for 35 min using the TwistAmp nfo kit (TwistDx Ltd., UK). Primers (420 nM), probe (120 nM), rehydration buffer (29.5 μL), magnesium acetate (14 mM) and DNA template (2.5 μL, 0.5 nM) were combined in a 50 μL reaction volume. All the reagents, except for the DNA template and magnesium acetate, were prepared in a master mix, which was used to rehydrate the dried reaction pellets. The DNA template was added to the resulting mixture, and magnesium acetate was pipetted into the cap of each tube and was centrifuged down to initiate amplification. Subsequent RPA amplicons were purified by the addition of 100% ice-cold ethanol and incubation for 20 min on ice before centrifugation of precipitated nucleic acids (13500×g, 8 min), followed by a 70% ice-cold ethanol wash and re-centrifugation (13500×g, 8 min). Pellets were re-suspended in TE buffer (Tris 10 mM, EDTA 0.1 mM, pH 8.0) to the original RPA volume of 50 μL.

Multiplex RPA Amplification

The RPA reaction was conducted at 39° C. for 35 min using the TwistAmp nfo kit (TwistDx Ltd., UK). For numerical displays (Table 9), the 50 μL reaction volume contained either: (i) for numbers “0”, “2”, “3”, “5”, “6”, “9” and “8”: each forward primer is (140 nM; except for Biotin at 280 nM), reverse primer (840 nM), probe (240 nM), rehydration buffer (29.5 μL), magnesium acetate (14 mM) and DNA template (2.5 μL, 0.5 nM); or (ii) for numbers “1”, “4” and “7”: each forward primer (140 nM; except for Biotin 280 nM), reverse primer (420 nM), probe (120 nM), rehydration buffer (29.5 μL), magnesium acetate (14 mM) and DNA template (2.5 μL, 0.5 nM). Amplicons were again purified by ethanol precipitation as described for the single-plex reactions.

TABLE 9
Forward primer 5′ molecular labels
for each multiplex RPA numerical display

| Number | Tags labelled at 5′ of forward primers |
|---|---|
| 0 | Biotin, Digoxigenin (NHS Ester), TAMRA ™ (NHS Ester), |
| | Texas Red ®-X (NHS Ester)ᵣ DNP-X C6—NH and Dansyl-X |
| | C6—NH |
| 1 | Biotin and TAMRA ™ (NHS Ester) |
| 2 | Digoxigenin (NHS Ester), TAMRA ™ (NHS Ester), Cascade |
| | Blue C6—NH, DNP-X C6—NH and Dansyl-X C6—NH |
| 3 | Biotin, Digoxigenin (NHS Ester), TAMRA ™ (NHS Ester), |
| | Cascade Blue C6—NH and Dansyl-X C6—NH |
| 4 | Biotin, TAMRA ™ (NHS Ester), Texas Red ®-X (NHS Ester), |
| | and Cascade Blue C6—NH |
| 5 | Biotin, TAMRA ™ (NHS Ester), Texas Red ®-X (NHS Ester), |
| | Cascade Blue C6—NH and Dansyl- X C6—NH |
| 6 | Bio-tin, Digoxigenin (NHS Ester), Texas Red ®-X (NHS Ester), |
| | Cascade Blue C6—NH, DNP-X C6-NH and Dansyl-X C6—NH |
| 7 | Biotin, Digoxigenin (NHS Ester), TAMRA ™ (NHS Ester) |
| 8 | Biotin, Digoxigenin (NHS Ester), TAMRA ™ (NHS Ester), |
| | Texas Red ®-X (NHS Ester), Cascade Blue C6—NH, DNP-X |
| | C6—NH Dansyl-X C6—NH |
| 9 | Biotin, Digoxigenin (NHS Ester), TAMRA ™ (NHS Ester), |
| | Texas Red ®-X (NHS Ester), Cascade Blue C6—NH and |
| | Dansyl-X C6—NH |

Preparation of AuNP Conjugates

Anti-fluorescein antibody was coupled to AuNPs which served as the signaling molecule (red in colour) to allow the visualisation of the immuno-sandwich complex by eye observation. Coupling to AuNPs (40 nm, 20 OD/vial, which is equivalent to 9×1010 particles per vial in 50 μL) was performed using the InnovaCoat® GOLD 10× Multi Explorer labelling kit (BioNovus Life Sciences, Cherrybrook, NSW, Australia). Briefly, reagents were thawed to 25° C. and 12 μL antibody (diluted to 0.1 mg/mL using the diluent provided) was mixed with 42 μL reaction buffer. The mixture (45 μL) was used to resuspend a vial of InnovaCoat GOLD nanoparticles which was incubated for 10 min before addition of 5 μL Quencher, resulting in a final 20 OD solution (50 μL) of anti-fluorescein/AuNP. The conjugates were washed twice with the borate running buffer by centrifuging at 14500×g for 6 min, before resuspension to the original (50 μL) volume. Conjugates were stored at 4° C.

Preparation of Single-Plex Lateral Flow and Multiplex Lateral Flow Strips

Conjugate and sample pads (Millipore, Billerica, Mass., USA) were blocked with blocking solution (1% polyvinyl alcohol, 20 mM Tris base, pH 7.4) for 30 min and dried at room temperature for 2 h. The two pads were soaked in borate running buffer (100 mM H₃BO³, 100 mM Na₂B₄O₇, 1% BSA, 0.05% Tween 20, pH 8.8) for 30 min before drying at 25° C. for 6 h.

Assembled devices (6.1 cm×0.3 cm) for single-plex lateral flow strips comprised treated sample pad (1.5 cm), treated conjugate pad (0.6 cm), a nitrocellulose membrane (2.5 cm; Hi-Flow Plus HF135) and an absorbent pad (1.5 cm) (Millipore, Billerica, Mass., USA) all combined on an adhesive backing card (KENOSHA c.v., Schweitzerlaan, Amstelveen, Netherlands), with a 0.1 cm overlap between components. The multiplex lateral flow strips were assembled using the same components and procedure, using treated sample pad (0.5 cm), treated conjugate pad (0.6 cm), a nitrocellulose membrane (3.5 cm) and an absorbent pad (2.5 cm).

For single-plex lateral flow detection, antibodies [either anti-biotin (1.0 mg/mL), anti-Digoxiginin (0.75 U/μL), anti-TAMRA (1.0 mg/mL), anti-Texas Red (1.0 mg/mL), anti-Alexa Fluor® 405/Cascade Blue® (3.0 mg/mL), anti-Dinitrophenyl-KLH (2.0 mg/mL) or anti-Dansyl (1.0 mg/mL)] were pipetted (0.4 μL) onto the test zone of the nitrocellulose membrane. Rabbit anti-mouse antibody (1 mg/mL in 50% glycerol) was pipetted (0.4 μL) at the control zone. Test and control antibodies were spotted 0.5 cm apart and dried at 25° C. for 45 min. For multiplex lateral flow detection, each detection antibodies (0.2 μL) were deposited and, as a control, rabbit anti-mouse antibody was pipetted (0.2 μL) in triplicate at the end of each array.

Single-Plex Lateral Flow and Multiplex Lateral Flow Test Procedures

Single-plex lateral flow detection was performed as described previously¹² but used purified RPA amplicons. Briefly, anti-fluorescein/AuNP conjugate (1 μL) was pipetted onto the conjugate pad and the strip was dipped into a mixture containing 100 μL running buffer and 5 μL of purified RPA amplicon. An additional 1 μL anti-fluorescein/AuNP conjugate was pipetted onto the conjugate pad once the running buffer reached the bottom of the absorbent pad as this double-run method has been demonstrated to be effective for developing high signal intensity with reduced anti-fluorescein/AuNP consumption¹². The strip was developed for 15 min. All experiments were repeated at least three times to demonstrate consistency of results.

For the single-plex RPA combined with multiplex lateral flow detection, purified RPA amplicons (2.0 μL, 5′ FAM/3′ X, X=Digoxigenin N, 6-TAMRASp, Texas Red-XN, Cascade Blue C6-NH or DNP-X C6-NH) and/or purified RPA amplicons (3.0 μL, 5′ FAM/3′ X, X=Biotin or Dansyl-X C6-NH) were mixed with 220 μL running buffer. Briefly, anti-fluorescein/AuNP conjugate (2.5 μL) was pipetted onto the conjugate pad and the strip was dipped into the RPA amplicon and running buffer mixture. An additional 2.5 μL anti-fluorescein/AuNP conjugate was pipetted onto the conjugate pad once the running buffer reached the bottom of the absorbent pad. The strip was developed for 25 min. All experiments were repeated at least twice to demonstrate consistency of results.

For the multiplex RPA in combination with multiplex lateral flow detection, anti-fluorescein/AuNP conjugate (2.5 μL) was pipetted onto the conjugate pad and the strip was dipped into a mixture containing 220 μL running buffer and 5 μL of purified RPA amplicon (corresponding to display numbers 0 to 9). An additional 2.5 μL anti-fluorescein/AuNP conjugate was pipetted onto the conjugate pad once the running buffer reached the bottom of the absorbent pad. The strip was developed for 25 min. All experiments were repeated at least twice to demonstrate consistency of results.

Image Analysis

Reacted lateral flow strips were dried, imaged using the MultiDoc-ItTM Digital Imaging System (Upland, Calif., USA), and analysed using ImageJ software (National Institutes of Health, Md., USA). Image brightness/contrast and colour balance were auto-adjusted.

### Results

The present inventors chose Biotin, Digoxigenin (NHS Ester), TAMRA™ (NHS Ester), Texas Red®-X (NHS Ester), Cascade Blue C6-NH, DNP-X C6-NH or Dansyl-X C6-NH to be incorporated as 5′ labels on the forward primer. During the RPA reaction these labels became combined with a 5′ Fluorescein amidite (FAM) labelled probe, to obtain dual-labelled double-stranded amplicons followed by single-plex LFD detection (FIG. 7).

FIG. 7: RPA and single-plex lateral flow sandwich assay. A: During the RPA reaction (i) the 5′ labelled primer, reverse primer and the 5′ FAM labelled TwistAmp™ LF probe bind to DNA, and (ii) the 3′ block on the probe is removed by the enzyme nfo, allowing (iii) extension by Bst polymerase to create (iv) a dual-labelled double-stranded amplicon. B: Single-plex lateral flow detection captures the dual-labelled RPA amplicon via antibody-hapten binding. Incorporation of AuNPs on the capture antibody (mouse anti-fluorescein antibody) enables visualisation of binding through aggregation of the AuNP at the test dot, which produces a red colour. Rabbit anti-mouse antibody is deposited in parallel as a control, and directly binds the capture mouse anti fluorescein capture antibody. C. Single-plex trial of demonstration successful RPA incorporation of seven RPA forward primer 5′ labels and subsequent detection on single _{A}lex LFD via corresponding anti-label antibodies. Reactions were performed with (+) or without (−) DNA template, and repeated at least twice. A representative photograph is shown.

Importantly, the DNA template was kept identical in each case to eliminate any recombinase preference bias that different DNA sequences might demonstrate during the RPA reaction. Successful incorporation of the labelled primer was observed by the appearance of a coloured LFD test dot when DNA template was supplied into the RPA reaction, with no reactions in the absence of DNA template (FIG. 7C). This is the first report of successful RPA incorporation of primers containing TAMRA, Texas Red, Cascade Blue, DNP and Dansyl. Previously, Crannell et al.¹⁴ reported a failure of RPA incorporation of five different 5′ labels (Cy5, Cy3, bromodeoxyuridine, tetrachlorofluorescein and hexachlorofluorescein) compared to successful hybridisation with three 5′ labels. The successful incorporation of seven labels during RPA-LFD detection here suggests that RPA generally tolerates 5′ label incorporation, despite the aforementioned report that five different 5′ labels[5] were not successfully detected using a RPA-LFD detection.

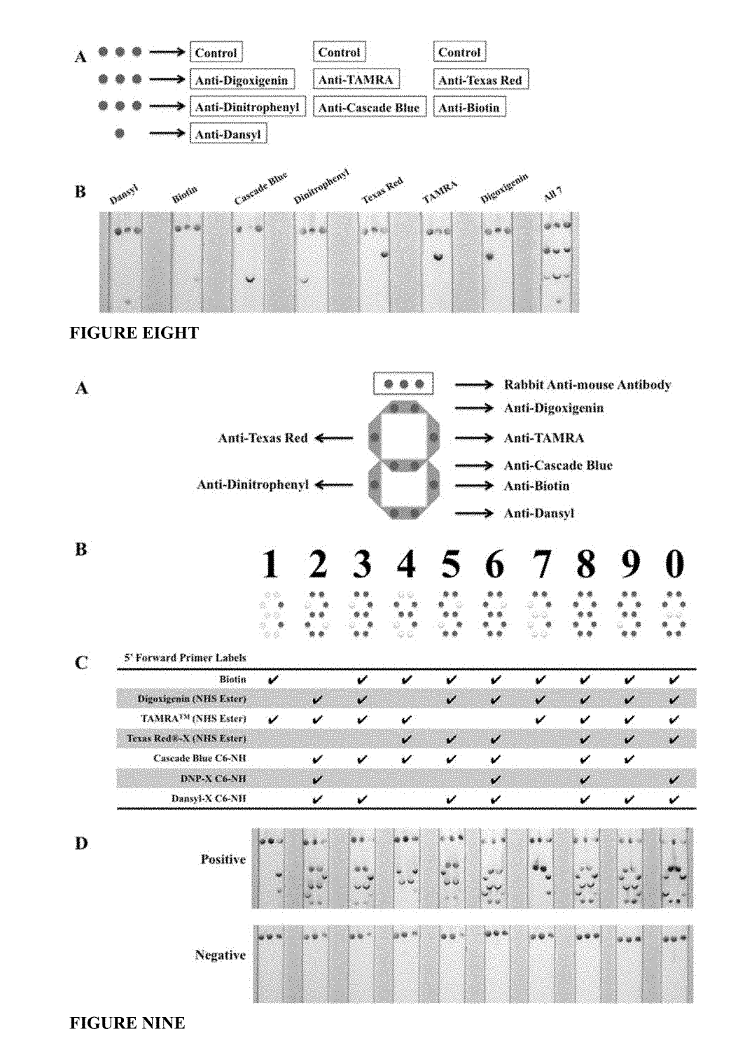

To determine the capacity for multiplex LFD detection, antibodies that detect the seven successful RPA labels were deposited in a multiplex LFD device and tested for specificity of detection for each individual RPA reaction. Results showed that all the seven RPA dual-labelled amplicons only produced a coloured test dot at the position corresponding to the antibody specific to their 5′ forward primer molecular label, indicating specific detection in each case (FIG. 8).

FIG. 8: Results of single-plex RPA in combination with multiplexed lateral flow detection. Specificity was determined by depositing anti-label antibodies on a LFD (top) and depositing each dual-labelled PRA amplicon (5 μL; with 5′ FAM and 3′ X, X=Biotin, Digoxigenin (NHS Ester), TAMRA™ (NHS Ester), Texas Red®-X (NHS Ester), Cascade Blue C6-NH, DNP-X C6-NH or Dansyl-X C6-NH). In the last panel (all 7) multiplex detection of a mixture of all RPA reactions (3.0 μL of 5′ FAM/3′ Biotin and 5′ FAM/3′ Dansyl-X C6-NH, and 2.0 μL of 5′ FAM/3′ X, X=Digoxigenin_N, 6-TAMRASp, Texas Red-XN, Cascade Blue C6-NH or DNP-X C6-NH). Reactions were repeated at least three times; a representative photograph is shown.

It was then determined the capacity of the multiplex LFD to detect mixed RPA-amplified products. These results indicated that all seven RPA dual-labelled amplicons could be detected simultaneously on a single LFD (FIG. 8). However, it was found that extra volumes of 5′ FAM/3′ Biotin and 5′ FAM/3′ Dansyl-X C6-NH (3.0 μL instead of 2.0 μL for the other RPA dual-labelled amplicons) were needed in the sample mixture to achieve approximately equal test dot intensities for all the seven antigen-antibody detections on multiplexed lateral flow detection. It is likely that the binding affinities of biotin to anti-biotin and dansyl-X C6-NH to anti-dansyl were weaker in comparison to the other antigen-antibody combinations, as suggested by the intensity of the test dot during the single-plex lateral flow detection results (FIG. 7C).

Successful operation of the single-plex RPA combined with multiplex LFD encouraged us to trial multiplex RPA detection, by combining forward primers labelled with different tags in a single tube (with reverse primer, probe and other RPA reagents). Again, the present inventors continued to use an identical DNA template to eliminate any binding bias towards the DNA template among the labelled primers during RPA reaction. Results indicated that the forward primers labelled with different tags (except for the biotin tag) showed approximately equal incorporation efficiencies into the DNA template (FIG. 9).

FIG. 9: Results of multiplex RPA in combination with multiplex lateral flow detection. A: Positioning of the detection antibodies to form the 7-segments of the display. B: Required number displays. C: Addition of labelled RPA forward primer signature mixtures (5′ labelled with Biotin, Digoxigenin (NHS Ester), TAMRA™ (NHS Ester), Texas Red®-X (NHS Ester), Cascade Blue C6-NH, DNP-X C6-NH or Dansyl-X C6-NH) with reverse primer, probe and other RPA reagents in one reaction tube for defined number displays. D: Resulting successful appearance of numbers (0 to 9) on the lateral flow strips. The assay was performed at least three times with similar results; a photograph of one test is shown.

However, a double concentration of the 5′ biotin labelled forward primer was required in comparison to the other labelled forward primers in order to form dual-labelled amplicons that could be visualised on the multiplexed LFD. Any further increases in the concentration of 5′ biotin labelled primer interfered with the binding efficiency of 5′ Dansyl-X C6-NH labelled primer to the DNA template and reduced test dot intensity of Dansyl label pair on the multiplex display of number 8. It is noted that the antibody deposition (of the test dots) was performed by hand pipetting, which led to the appearance of comet tails for some of the test dots. This issue could be solved using advanced deposition techniques such as inkjet printing and optimisation of printing parameters (e.g. surface tension and viscosity)¹⁵.

The combination of RPA with the colourimetric 7-segment display offers significant advantages over general multiplex nucleic acid amplification coupled with multiplexed LFDD¹⁶. The demonstration herein of detection technology is faster (35 min in comparison to 1 h PCR), does not require probe hybridisations before the LFD detection, and produces a colourimetric signal that can be inspected by eyes without external readers. The multiplexing RPA system using seven labelled forward primers in a single tube, combined with binary encoding, dramatically increases multiplexing potential. In this regard, the numbers display demonstrated herein represents an unbiased test of different label combinations. The seven different labels have the potential to be uniquely combined to produce 127 (27−1) different display patterns using defined combinations of RPA dual-labelled amplicons (a form of barcoding). However, testing each combination would be an exhaustive and costly exercise. By applying these combinations to produce a 7-segment display, the present inventors sample and demonstrate success for a smaller test set of 10 different combinations. Such sampling testing strategies are common in systems with complex decision trees such as integrated circuits, and formal sampling strategies will need to be adopted for LFD detection as system complexity increases.

In summary, this study provides for the successful incorporation of seven 5′ labelled primers during single-tube RPA followed by multiplexed LFD detection, demonstrating for the first time that TAMRA™ (NHS Ester), Texas Red®-X (NHS Ester), Cascade Blue C6-NH, DNP-X C6-NH and Dansyl-X C6-NH can be incorporated as 5′ labels during RPA. In addition, molecular and binary encoding was used to demonstrate the first intuitive LFD result display to be coupled with any nucleic acid amplification reactions. The intuitive display is highly relevant for POC applications, because it provides easy results interpretation compared to detecting multiple lines or dots, does not require special equipment for signal visualisation, and provides information-compact results on a lateral flow device, where space and time are premium commodities.

### REFERENCES

1. Marchant J, editor Next generation molecular and point-of-care diagnostics driving personalized healthcare. Innovations in Diagnostics; 2005; London, England: Business Insights Limited.

2. Chard T. Pregnancy tests: a review. Hum Reprod. 1992 May 1, 1992;7(5):701-10.

3. Devices B-AM. “Point of Care diagnostic market expected to be worth over $20 billion by 2014”: Bio-Alternative Medical Devices; [cited 2015 Jun. 22nd, 2015]. Available from: http://www.bioamd.com/investors.

4. I. Bio-Rad Laboratories, Vol. 2015, Bio-Rad Laboratories, Inc., 2015.

5. L. Corporation, Vol. 2015, Luminex Corporation., 2015.

6. Tian L, Sato T, Niwa K, Kawase M, Tanner A C, Takahashi N. Rapid and sensitive PCR-dipstick DNA chromatography for multiplex analysis of the oral microbiota. Biomed Res Int. 2014; 2014:180323. PubMed PMID: 25485279. Pubmed Central PMCID: PMC4251647. Epub 2014/12/09. eng.

7. Washburn EW. The dynamics of capillary flow. Phys Rev. 1921;17(3):273-83.

8. Becton DaC. BD™ EZ Flu A+B Test: Becton, Dickinson and Company; 2014 [cited 2015 Jan. 12th]. Available from: http://www.bd.com/ds/product Center/256050.asp.

9. Alere. Alere BinaxNOW® Influenza A&B Card: Alere; 2010-2015 [cited 2015 Jan. 13th]. Available from: http://www.alere.com/us/en/product-details/binaxnow-influenza-a-and-b.html.

10. Technologies A. RAID: Alexeter Technologies; 2014 [cited 2015 Jan. 12th]. Available from: http://www.alexeter.com/biow/Products/products/strips/RAID_DX.asp.

11. S. M. Hossain, J. D. Brennan, Anal. Chem. 2011, 83, 8772-8778; bC. Z. Li, K. Vandenberg, S. Prabhulkar, X. Zhu, L. Schneper, K. Methee, C. J. Rosser, E. Almeide, Biosens. Bioelectron. 2011, 26, 4342-4348; cW. Wang, W. Y. Wu, W. Wang, J. J. Zhu, J. Chromatogr. A 2010, 1217, 3896-3899.

12. J. Li, D. McMillan, J. Macdonald, Sensor. Mater. 2015, 27, 549-561.

13. Y. Yang, X. Qin, G. Wang, J. Jin, Y. Shang, Z. Zhang, Virol. J. 2016, 13, 46.

14. Z. Crannell, A. Castellanos-Gonzalez, G. Nair, R. Mejia, A. C. White, R. Richards-Kortum, Anal. Chem. 2016.

15. J. Li, F. Rossignol, J. Macdonald, Lab Chip. 2015, 15, 2538-2558.

16. aY. Xu, Y. Liu, Y. Wu, X. Xia, Y. Liao, Q. Li, Anal. Chem. 2014, 86, 5611-5614; bI. K. Litos, P. C. Ioannou, T. K. Christopoulos, J. Traeger-Synodinos, E. Kanavakis, Biosens. Bioelectron. 2009, 24, 3135-3139; cL. Tian, T. Sato, K. Niwa, M. Kawase, A. C. Tanner, N. Takahashi, Biomed. Res. Int. 2014, 2014, 180323; dP. Noguera, G. A. Posthuma-Trumpie, M. van Tuil, F. J. van der Wal, A. de Boer, A. P. H. A. Moers, A. van Amerongen, Anal Bioanal Chem 2011, 399, 831-838; eM. Blaz̆ková, M. Koets, P. Rauch, A. Amerongen, Eur. Food Res. Technol. 2009, 229, 867-874; fA. B. Nurul Najian, E. A. Engku Nur Syafirah, N. Ismail, M. Mohamed, C. Y. Yean, Anal. Chim. Acta. 2016, 903, 142-148.

1. A lateral flow device comprising three binding molecule populations for detection of multiple analytes, wherein each said binding molecule population:
has binding specificity for a different type of target ligand,
has less than: 10%, 5%, 4%, 3%, 2%, or 1%, cross-reactivity with a target ligand for which any other said binding molecule population has binding specificity; and
is capable of contributing to the formation of a signalling complex capable of providing a detectable signal only in the presence of a target analyte, wherein the signalling complex comprises a signalling molecule, and a member of any one of said binding molecule populations bound directly to said target ligand for which the member has binding specificity, which is in turn bound either directly or indirectly to said target analyte.

2. The lateral flow device according to claim 1, wherein each said binding molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin and biotin.

3. The lateral flow device according to claim 1 or claim 2, wherein each of said multiple analytes, or each said binding molecule population is immobilised within a detection zone of the device in a spatially separated arrangement.

4. The lateral flow device according to claim 3, wherein
the binding molecule population that exhibits the lowest level of sensitivity in the presence of its target analyte is positioned closer to a sample application zone of the device compared to any other binding molecule population immobilised in the detection zone.

5. The lateral flow device according to claim 3, wherein
the binding molecule population that exhibits the highest level of sensitivity in the presence of its target analyte is positioned furthest from a sample application zone of the device compared to any other analyte immobilised in the detection zone.

6. The lateral flow device according to any one of claims 3 to 5, wherein the binding molecule populations are selected according to their sensitivity level and cross reactivity characteristics, and spatially positioned on the device according to forming predetermined patterns in a detection zone of the device should specific binding occur.

7. The lateral flow device according to any one of claims 3 to 5, wherein the spatially separated arrangement is non-linear, a non-linear dot format, or a line format.

8. The lateral flow device according to any one of claims 3 to 7, wherein the spatially separated arrangement is a dot matrix format.

9. The lateral flow device according to any one of claims 3 to 8, comprising seven different detection molecule populations each immobilised within a detection zone of the device in a dot matrix format, wherein
each of three of the binding molecule populations are represented in the dot matrix by two dots per population which are equidistant or substantially equidistant from a sample application zone of the device, and
each of four of the binding molecule populations are represented in the dot matrix by one dot per population which are at different distances from a sample application zone of the device, and
all said dots are collectively arranged in a pattern forming the digit eight.

10. The lateral flow device according to any one of claims 1 to 9, wherein the three binding molecule populations are a combination shown in Table 3.

11. The lateral flow device according to any one of claims 1 to 10 comprising four binding molecule populations, wherein the four binding molecule populations are a combination shown in Table 4.

12. The lateral flow device according to any one of claims 1 to 11 comprising five binding molecule populations, wherein the five binding molecule populations are a combination shown in Table 5.

13. The lateral flow device according to any one of claims 1 to 12 comprising six binding molecule populations, wherein the six binding molecule populations are a combination shown in Table 6.

14. The lateral flow device according to any one of claims 1 to 13 comprising seven binding molecule populations, wherein the seven binding molecule populations are a combination shown in Table 7.

15. The lateral flow device according to claim 14, wherein the seven detection molecule populations are anti-digoxigenin antibodies, anti-TAMRA antibodies; anti-Texas Red antibodies, anti-dinitrophenyl antibodies, anti-Cascade Blue antibodies, either one of streptavidin or anti-biotin antibodies, and either one of anti-Dansyl antibodies or anti-Cy5 antibodies.

16. The lateral flow device according to any one of claims 1 to 15, further comprising a positive control molecule population.

17. The lateral flow device according to any one of claims 1 to 16, further comprising a capture molecule population having binding specificity for a capture ligand, wherein,
the capture molecule population and each said binding molecule population have binding specificity for different target ligands;
the capture molecule population has less than: 10%, 5%, 4%, 3%, 2%, or 1%, cross-reactivity with a target ligand for which any other said binding molecule population has binding specificity;
individual members of the capture molecule population are each bound to a signal generating molecule capable of providing said detectable signal; and
said signal complex comprises a member of the capture molecule population bound to said capture ligand which is in turn bound to said target analyte.

18. The lateral flow device according to claim 16, wherein the capture molecule population is selected from the group consisting of: anti-digoxigenin antibodies, anti-dinitrophenyl antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, biotin, and streptavidin.

19. The lateral flow device according to claim 17 or claim 18, wherein
the positive control molecule population has binding specificity for each said binding molecule population,
individual members of the positive control molecule population are each bound to a signal generating molecule capable of providing a detectable control signal,
said individual members of the positive control molecule population are bound to the same type of signal generating molecule as said individual members of the capture molecule population, and
said individual members of the positive control molecule population and said individual members of the capture molecule population are each bound to distinct signal generating molecules.

20. The lateral flow device according to any one of claims 1 to 15, wherein individual members of each said binding molecule population are bound to a signal generating molecule capable of providing said detectable signal.

21. The lateral flow device according to claim 20, wherein
the positive control molecule population comprises known quantities of said multiple analyte populations immobilised within a detection zone of the device in a spatially separated arrangement, and
said positive control molecule population is capable of providing a detectable control signal when bound to said signal generating molecule.

22. The lateral flow device according to any one of claims 1 to 15, wherein the multiple analytes are each bound to a signal generating molecule capable of providing said detectable signal.

23. The lateral flow device according to claim 22, wherein
the positive control molecule population comprises known quantities of said multiple analyte populations each bound to a signal generating molecule capable of providing a detectable control signal.

24. The lateral flow device according to any one of claims 20 to 23 comprising eight or more binding molecule populations selected from: anti-digoxigenin antibodies, anti-TAMRA antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, biotin, and streptavidin.

25. The lateral flow device according to any one of claims 1 to 24, wherein the positive control molecule population and/or the capture molecule population is/are present in a conjugate zone of the device.

26. The lateral flow device according to any one of claims 1 to 25 comprising any one or more of: a membrane, a sample pad, a conjugate pad, an absorbent pad, an incubation pad, a detection pad, running buffer, and/or plastic housing.

27. The lateral flow device according to any one of claims 1 to 26 comprising any one or more of:
a membrane produced from any one or more of nitrocellulose, nylon, polyethersulfone, polyethylene, polyvinylidine difluoride (PVDF), fused silica;
a series of interconnected pads comprising any one or more of: a sample pad for distribution of sample solution to upstream components; a conjugate pad adjacent to the sample pad for controlling release of reactants onto the membrane; an absorbent pad at or close proximity to the base of the lateral flow device for enhancing the capillary driving force and absorbing any unreacted substances; an incubation pad and/or a detection pad adhered to a surface of the membrane for stabilisation of the membrane,
running buffer selected from phosphate-buffered saline (PBS), tris-buffered saline), borate, and buffers comprising blockers including casein, bovine serum albumin (BSA), PVA, and
plastic housing for sealing the device, comprising a sample application inlet and a window above the detection zone.

28. A method for multiplex lateral flow detection of different target analyte populations in a sample, the method comprising:
labelling each target analyte population in the sample with a single type of ligand selected from the group consisting of: digoxigenin, tetramethylrhodamine (TAMRA), dinitrophenyl, Texas Red, cascade blue, streptavidin, biotin, Cy5, dansyl, and fluorescein;
applying the sample to the lateral flow device according to any one of claims 1 to 27, and
determining whether one or more individually detectable signals are generated
wherein each individually detectable signal generated is dependent on and indicative of the presence of a specific target analyte population in the sample,
and wherein each said target analyte population for detection in the method is labelled with a different ligand compared to all other target analyte populations, and each said different ligand can contribute to the induction of an individually detectable signal in the lateral flow device.

29. A method for determining an absence of different target analyte populations in a sample by multiplex lateral flow detection, the method comprising:
labelling each target analyte population in the sample with a single type of ligand selected from the group consisting of: digoxigenin, tetramethylrhodamine (TAMRA), Texas Red, dinitrophenyl, cascade blue, streptavidin, biotin, Cy5, dansyl, and fluorescein;
applying the sample to the lateral flow device according to any one of claims 1 to 27, and
determining whether one or more individually detectable signals dependent on the presence of a specific target analyte population in the sample are generated,
wherein failure to detect a given signal is indicative of a specific target analyte population being absent in the sample,
and wherein each said target analyte population for detection in the method is labelled with a different ligand compared to all other target analyte populations, and each said different ligand can contribute to the induction of an individually detectable signal in the lateral flow device.

30. The method according to claim 28 or claim 29, wherein the target analyte populations are nucleic acids, proteins, peptides, lipids, small molecules, or any combination thereof.

31. The method according to any one of claims 28 to 30, wherein the target analyte populations are nucleic acids.

32. The method according to claim 30 or claim 31, wherein the nucleic acids are DNA.

33. The method according to claim 31 or claim 32, wherein said labelling each target analyte population in the sample comprises: polymerase chain reaction (PCR), isothermal nucleic acid amplification, or a combination thereof.

34. The method according to claim 33, wherein the isothermal nucleic acid amplification is selected from any one or more of: LAMP, HDA, NASBA, RPA, RT-PCR or any combination thereof.

35. The method according to any one of claims 31 to 33, wherein said labelling each target analyte population in the sample comprises two or more of PCR, RPA, LAMP, HDA NASBA.

36. The method according to any one of claims 28 to 30, wherein at least one of said target analyte populations comprises proteins, peptides, lipids or small molecules, and said labelling of said target analyte comprises use of aptamers and/or antibodies having binding specificity for members of said at least one target analyte population and are each bound to said single type of ligand.

37. The method according to any one of claims 28 to 36, wherein:
the target analyte populations are nucleic acids,
said single type of ligand selected from the group consisting of: digoxigenin, Texas Red, dinitrophenyl, cascade blue, biotin, CyS, dansyl, and fluorescein, is bound to a first terminus of each nucleic acid,
a second terminus of each said nucleic acid is labelled with a ligand selected from the group consisting of: digoxigenin, Texas Red, dinitrophenyl, cascade blue, biotin, Cy5, dansyl, and fluorescein, is bound to a first terminus of each nucleic acid,
the ligand bound to the first terminus is a different type of ligand to that which is bound to the second terminus, and
the ligand bound to the second terminus is the same in nucleic acids of all target analyte populations.

38. The method according to any one of claims 28 to 37, wherein the detectable signal is a colourimetric signal including a signal generated from enzymes or enzyme substrates, beads, particles, fluorescent dyes, nanomaterials (e.g. latex beads or colloidal gold particles, carbon particles, magnetic particles, paramagnetic particles, quantum dots, up-converting phosphorus, nano microspheres, nano-tubes, chelate-loaded silica, europium), liposomes, and fluorescent immunoliposomes.

39. A method for producing a lateral flow device, the method comprising depositing at least three binding molecule populations in a detection zone of the lateral flow device, wherein each said binding molecule population:
has binding specificity for a different target ligand;
has less than: 10%, 5%, 4%, 3%, 2%, or 1%, cross-reactivity with a target ligand for which any other said binding molecule population has binding specificity;
is immobilised within a detection zone of the device, and spatially separated from all other detection molecule populations in the detection zone; and
is capable of contributing to the formation of a signalling complex capable of providing a detectable signal only in the presence of a target analyte, wherein the signal complex comprises a member of any one of said binding molecule populations bound to said target ligand for which the member has binding specificity, which is in turn bound to said target analyte.

40. The method according to claim 39, wherein each said binding molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin and biotin.

41. The lateral flow device according to claim 39 or claim 40 wherein
the binding molecule that exhibits the lowest level of sensitivity in the presence of its target analyte is positioned closer to a sample application zone of the device compared to any other binding molecule population immobilised in the detection zone.

42. The lateral flow device according to any one of claims 39 to 41, wherein
the binding molecule that exhibits the highest level of sensitivity in the presence of its target analyte is positioned furthest from a sample application zone of the device compared to any other analyte immobilised in the detection zone.

43. The method according to any one of claims 39 to 42, wherein the spatially separated arrangement is any one or more of: non-linear, a non-linear dot or line format and/or a dot matrix format.

44. The method according to any one of claims 39 to 43 comprising depositing seven different detection molecule populations, wherein
each is immobilised within the detection zone of the device in a dot matrix format, wherein
each of three of the binding molecule populations are represented in the dot matrix by two dots per population which are equidistant or substantially equidistant from a sample application zone of the device, and
each of four of the binding molecule populations are represented in the dot matrix by one dot per population which are at different distances from a sample application zone of the device, and
all said dots are collectively arranged in a pattern forming the digit eight.

45. The method according to any one of claims 39 to 44, wherein the three binding molecule populations are a combination shown in Table 3.

46. The method according to any one of claims 39 to 45 comprising depositing four binding molecule populations in the detection zone, wherein the four binding molecule populations are a combination shown in Table 4.

47. The method according to any one of claims 39 to 46 comprising depositing five binding molecule populations in the detection zone, wherein the five binding molecule populations are a combination shown in Table 5.

48. The lateral flow device according to any one of claims 39 to 47 comprising depositing six binding molecule populations in the detection zone, wherein the six binding molecule populations are a combination shown in Table 6.

49. The method according to any one of claims 39 to 48, comprising depositing seven binding molecule populations in the detection zone, wherein the seven binding molecule populations are a combination shown in Table 7.

50. The method according to claim 49, wherein the seven detection molecule populations are anti-digoxigenin antibodies, anti-TAMRA antibodies; anti-Texas Red antibodies, anti-TAMRA antibodies, anti-Cascade Blue antibodies, either one of streptavidin or anti-biotin antibodies, and either one of anti-Dansyl antibodies or anti-Cy5 antibodies.

51. The method according to any one of claims 39 to 50, further comprising including a positive control molecule population in the device.

52. The method according to any one of claims 39 to 51, further comprising including a capture molecule population in the device having binding specificity for a capture ligand, wherein,
the capture molecule population and each said binding molecule population have binding specificity for different target ligands;
the capture molecule population has less than: 10%, 5%, 4%, 3%, 2%, or 1%, cross-reactivity with a target ligand for which any other said binding molecule population has binding specificity;
individual members of the capture molecule population are each bound to a signal generating molecule capable of providing said detectable signal; and
said signal complex comprises a member of the capture molecule population bound to said capture ligand which is in turn bound to said target analyte.

53. The method according to claim 52, wherein the capture molecule population is selected from the group consisting of: anti-digoxigenin antibodies, anti-tetramethylrhodamine (TAMRA) antibodies, anti-Texas Red antibodies, anti-dinitrophenyl antibodies, anti-cascade blue antibodies, anti-streptavidin antibodies, anti-biotin antibodies, anti-Cy5 antibodies, anti-dansyl antibodies, anti-fluorescein antibodies, streptavidin, and biotin.

54. The method according to claim 52 or claim 53, wherein
the positive control molecule population has binding specificity for each said binding molecule population,
individual members of the positive control molecule population are each bound to a signal generating molecule capable of providing a detectable control signal,
said individual members of the positive control molecule population are bound to the same type of signal generating molecule as said individual members of the capture molecule population, and
said individual members of the positive control molecule population and said individual members of the capture molecule population are each bound to distinct signal generating molecules.

55. The method according to any one of claims 39 to 54 comprising incorporating into the device any one or more of: a membrane, a sample pad, a conjugate pad, an absorbent pad, an incubation pad, a detection pad, running buffer, and/or plastic housing.

56. The method according to any one of claims 39 to 55 comprising incorporating into to the device any one or more of:
a membrane produced from any one or more of nitrocellulose, nylon, polyethersulfone, polyethylene, polyvinylidine difluoride (PVDF), fused silica;
a series of interconnected pads comprising any one or more of: a sample pad for distribution of sample solution to upstream components; a conjugate pad adjacent to the sample pad for controlling release of reactants onto the membrane; an absorbent pad at or close proximity to the base of the lateral flow device for enhancing the capillary driving force and absorbing any unreacted substances; an incubation pad and/or a detection pad adhered to a surface of the membrane for stabilisation of the membrane,
running buffer selected from phosphate-buffered saline (PBS) tris-buffered saline), borate, and buffers comprising blockers including casein, bovine serum albumin (BSA), PVA, and
plastic housing for sealing the device, comprising a sample application inlet and a window above the detection zone.

57. A lateral flow device obtained or obtainable by the method of any one of claims 39 to 56.
